(19) United States
(12) Patent Application Publication
(10) Pub. No.: US 2010/0130598 A1
(43) Pub. Date: May 27, 2010

(54) METHODS FOR INDUCING PROGRAMMED CELL DEATH

(75) Inventors: David Mathew Brown, North Ryde (AU); Alan James Husband, Pyrmont (AU); Gil Mor, Cheshire, CT (US); Ewan M. Tytler, Birmingham, AL (US)

Correspondence Address: MERCHANT & GOULD PC, P.O. BOX 2903, MINNEAPOLIS, MN, 55402-0903, US

(73) Assignee: Novogen Research Pty Ltd., North Ryde (AU)

(21) Appl. No.: 12/581,633

(22) Filed: Oct. 19, 2009

Related U.S. Application Data
(60) Provisional application No. 61/107,363, filed on Oct. 22, 2008.

(51) Int. Cl. A61K 31/352; C12N 5/071; A61P 35/00; A61P 37/06

(52) U.S. Cl. 514/456; 435/375

(57) ABSTRACT

The present invention relates to methods for inducing or promoting caspase-independent apoptosis in a cell, the method comprising exposing to the cell an effective amount of a compound of formula I as described herein. The invention also relates to methods for treating or preventing diseases and disorders by administering to subjects in need thereof an effective amount of a compound of formula I, wherein the compound induces or promotes caspase-independent apoptosis in at least one cell of the subject.

This application claims the benefit Under 35 U.S.C. §119(e) of U.S. Provisional Application No. 61/107,363, filed 22 Oct. 2008, the entire disclosure of which is incorporated herein by reference.

### FIELD OF THE INVENTION

The present invention relates generally to methods for inducing or promoting caspase-independent apoptosis and for inhibiting mTOR activity. The invention also relates to the treatment of diseases and conditions associated with aberrant/unwanted cell growth and/or proliferation.

### BACKGROUND OF THE INVENTION

Programmed cell death is an evolutionarily conserved pathway, the activation of which leads to an energy-dependent cell suicide mechanism. Two forms of programmed cell death are generally described in the literature, apoptosis or caspase-dependent cell death, and caspase-independent cell death. Caspase-dependent apoptosis is the better characterized pathway of the two types of programmed cell death such that the terms programmed cell death and apoptosis are typically used interchangeably. Caspase-mediated apoptosis involves the sequential activation of a group of proteases, the caspases, and can be activated either by ligation of death receptors like Fas and TNFR (extrinsic pathway), or by mitochondrial depolarization (intrinsic pathway). The Bcl₂ family of proteins, which has both pro-apoptotic (Bak, Bax, etc.) and anti-apoptotic members (Bcl₂, Bclₓ), controls mitochondrial integrity and the decision to engage the intrinsic pathway depends on the ratio of the pro-and anti-apoptotic members in the outer mitochondrial membrane.

Caspase-independent cell death encompasses events that occur when cells die in the absence of caspase activation. Autophagy is the most characterized caspase-independent programmed cell death pathway and is often called Type II programmed cell death. It involves the controlled formation of autophagosomes, double-membrane cytoplasmic vesicles, which can fuse with lysosomes thus leading to the digestion of molecules within the autophagosome. Autophagy is controlled by the Akt-mTOR pathway and involves key proteins such as Beclin-1 and Class III PI3 kinase. It is a pathway activated to promote cell survival, but due to the inherent mechanisms invoked can also lead to cell death. Other pathways for caspase-independent cell death, including caspase-independent apoptosis are reviewed in Hail et al, 2006.

Numerous studies have shown that most chemotherapy agents induce cell death by activating the apoptotic pathway and that resistance to apoptosis due to high intracellular levels of anti-apoptotic blockers like XIAP is a major cause of chemo-resistance. Indeed, molecular or drug targeting of apoptotic blockers like XIAP results in the reversal of chemo-resistance (see, for example, Alvero et al, 2006; Kluger et al, 2007). Aberrant cell growth and/or proliferation is also associated with a wide variety of disease conditions and there is increasing interest in the therapeutic application of apoptosis inducers.

As herein described, the present inventors have identified a class of isoflavonoid compounds which induce caspase-independent non-autophagic programmed cell death in human cells, thereby opening up a range of novel therapeutic avenues.

### SUMMARY OF THE INVENTION

According to a first aspect there is provided a method for inducing or promoting caspase-independent apoptosis in a cell, the method comprising exposing to the cell an effective amount of a compound of formula (I)

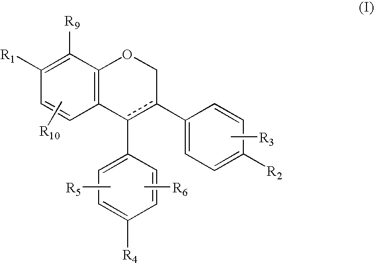

wherein
R₁ is hydrogen, hydroxy, alkyl, alkoxy, halo or OC(O)R₇,
R₂ and R₃ are independently hydrogen, hydroxy, alkoxy, alkyl, cycloalkyl, halo or OC(O)R₇,
R₄, R₅ and R₆ are independently hydrogen, hydroxy, alkoxy, alkyl, cycloalkyl, acyl, amino, C₁₋₄-alkylamino or di(C₁₋₄-alkyl)amino, OC(O)R₇ or OR₈,
R₇ is hydrogen, alkyl, cycloalkyl, aryl, arylalkyl or amino, and
R₈ is aryl or arylalkyl,
R₉ and R₁₀ are independently hydrogen, hydroxy, alkyl, alkoxy or halo, and the drawing [symbol] represents a single bond or a double bond,
   or a pharmaceutically acceptable salt or derivative thereof.

In one embodiment, the compound is 3-(4-hydroxyphenyl)-4-(4-methoxyphenyl)-8-methylchroman-7-ol, with the structure:

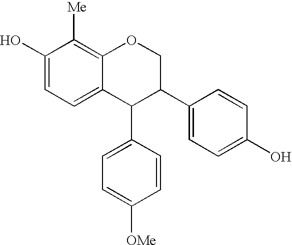

In an alternative embodiment, the compound is 3-(4-hydroxyphenyl)-4-(4-methoxyphenyl)chroman-7-ol, with the structure:

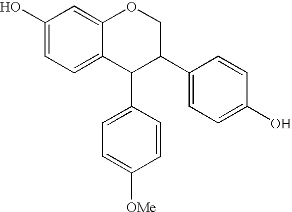

Exposure of the cell to the compound may occur in vitro, ex vivo or in vivo.

In a particular embodiment the cell is not a cancer cell. By way of example the cell may be a myocardial cell or immune cell. The immune cell may be a proliferating T cell.

According to a second aspect there is provided a method for inhibiting mTOR activity in a cell, the method comprising exposing to the cell an effective amount of a compound of formula (I) as described herein.

Typically the inhibition of mTOR activity comprises dephosphorylation of mTOR.

According to a third aspect there is provided a method for the treatment or prevention of a disease or condition, the method comprising administering to a subject in need thereof an effective amount of a compound of formula (I) as described herein, or a pharmaceutically acceptable salt or derivative thereof, optionally in association with one or more pharmaceutically acceptable diluents, adjuvants and/or excipients, wherein the compound induces or promotes caspase-independent apoptosis in at least one cell of the subject.

According to a fourth aspect there is provided a method for the treatment or prevention of a disease or condition, the method comprising administering to a subject in need thereof an effective amount of a compound of formula (I) as described herein, or a pharmaceutically acceptable salt or derivative thereof, optionally in association with one or more pharmaceutically acceptable diluents, adjuvants and/or excipients, wherein the compound inhibits mTOR activity in at least one cell of the subject.

In a particular embodiment in accordance with the third or fourth aspect the cell is not a cancer cell. By way of example the cell may be a myocardial cell or an immune cell.

Typically in accordance with the third or fourth aspect the disease or condition is associated with aberrant or otherwise unwanted cell growth or proliferation. In an embodiment, the disease or condition may be selected from stenosis or restenosis, transplant rejection or rheumatoid arthritis. Where the cell proliferation is T cell proliferation, the disease or condition may be selected from T cell leukemias, autoimmune diseases, and transplant or graft rejections such as graft versus host disease. Autoimmune diseases include, but are not limited to, cirrhosis, psoriasis, lupus, rheumatoid arthritis, Addison's disease, infectious mononucleosis, Sézary's syndrome and Epstein-Barr virus infection.

For the treatment of stenosis or restenosis the compound or a composition comprising the compound may be coated onto or otherwise incorporated into a stent for introduction into a coronary artery. The stent may be such that the compound or composition is eluted from the stent over a period of time so as to achieve the desired outcome.

In one embodiment, the compound is 3-(4-hydroxyphenyl)-4-(4-methoxyphenyl)-8-methylchroman-7-ol, with the structure:

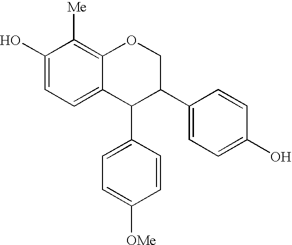

In an alternative embodiment, the compound is 3-(4-hydroxyphenyl)-4-(4-methoxyphenyl)chroman-7-ol, with the structure:

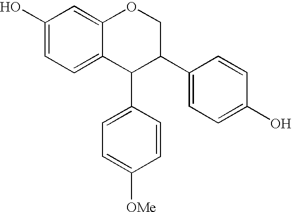

According to a fifth aspect there is provided an agent for the treatment or prevention of a disease or condition associated with aberrant or otherwise unwanted cell growth and/or proliferation, the agent comprising a compound of formula (I) as described herein, or a pharmaceutically acceptable salt or derivative thereof.

According to a sixth aspect there is provided the use of a compound of formula (I) as described herein for the manufacture of a medicament for inducing or promoting caspase-independent apoptosis in a cell.

According to a seventh aspect there is provided the use of a compound of formula (I) as described herein for the manufacture of a medicament for inhibiting mTOR activity in a cell.

According to an eighth aspect there is provided the use of a compound of formula (I) as described herein for the manufacture of a medicament for treating or preventing a disease or condition, wherein the compound induces or promotes caspase-independent apoptosis in at least one cell of the subject.

According to a ninth aspect there is provided the use of a compound of formula (I) as described herein for the manufacture of a medicament for treating or preventing a disease or condition, wherein the compound inhibits mTOR activity in at least one cell of the subject.

According to a tenth aspect there is provided an implantable medical device for delivering at least one active agent to a cell or tissue in a subject, wherein the at least one active agent comprises a compound of formula (I) as described herein.

Typically the compound is coated onto or otherwise incorporated into the device for administration of the compound to the cell or tissue. Optionally, the device also includes one or more additional active agents.

In an embodiment, the implantable medical device is a drug-eluting stent.

Typically in accordance with the above aspects and embodiments the subject is human. In other embodiments, the subject may be selected from the group consisting of, but not limited to: primate, ovine, bovine, canine, feline, porcine, equine and murine.

### BRIEF DESCRIPTION OF THE DRAWINGS

The present invention will now be described, by way of non-limiting example only, with reference to the accompanying drawings.

FIG. 1. (A) EOC cells were treated with increasing concentrations of Compound 1 for 24 h and cell viability determined as described herein. Results shown are representative of three independent experiments. (B) No-treatment control cells, and cells treated with Compound 1 (10 g/ml) for 24 h, were stained with Hoechst and PI and analyzed by flow cytometry. (C) Caspase activity was measured in cell lysates obtained from cells treated with increasing concentrations of Compound 1 or 2 μM Paclitaxel for 24 h.

FIG. 2. EOC cells were treated with 10 g/ml Compound 1 for the indicated time and whole cells lysates were analyzed by western blot for XIAP (A) and phospho-Akt (p-Akt) (B). β-actin and Akt blots demonstrate even loading. (C) Cells were treated with increasing concentrations of Compound 1 for 24 h in the presence or absence of Z-VAD-FMK (20 μM) or 3-MA (10 μM). These data are representative of those results obtained from all EOC cell lines analyzed.

FIG. 3. EOC cells were treated with 10 g/ml Compound 1 for the indicated time and whole cells lysates were analyzed by western blot for (A) phospho-mTOR and pS6k; (B) LC3-II; and (C) levels of 8 phospho-proteins as described herein. β-actin, mTOR, and S6k blots demonstrate even loading.

FIG. 4. A confocal microscope image of EOC cells either unstimulated (A) or treated with 10 g/ml Compound 1 for 2 h (B). Note the presence of intra-cellular vacoules in B but not in A (red arrows). Fluoresence microscope images of cells stained with JC-1; unstimulated (C) or treated with 10 μg/ml Compound 1 for 2 h (D). Rounded arrow points to punctuate red staining in unstimulated cells; diamond-ending arrow points to a cell with some mitochondrial depolarization; and arrowhead points to a cell with bright green fluorescence suggesting most mitochondria have depolarized.

FIG. 5. EOC cells were treated with 10 pg/ml Compound 1 for 1 h (A) and 4 h (B), stained with JC-1 dye, and analyzed by flow cytometry. Results are from a representative cell line. Similar results were obtained with other lines.

FIG. 6. (A) Western blot analysis of cell lysates and mitochondrial fractions prepared from EOC cells treated with 10 μg/ml Compound 1. Total cell lysates were analyzed for full-length Bid and mitochondrial fractions were analyzed for Beclin-1 and Bax. β-actin and VDAC are shown as loading controls. (B) Western blot analysis of anti-Beclin immunoprecipitates, derived from mitochondrial fractions of Compound 1 (10 μg/ml, 1 h) treated EOC cells, probed with anti-Bcl-2 and anti-Bak.

FIG. 7. EOC cells were treated with Compound 1 (10 μg/ml) for the indicated time and nuclear fractions prepared as described herein. Levels of AIF and EndoG were determined by western blot analyses. Topoisomerase I (Topo-I) is shown as loading control.

FIG. 8. EOC tumors were established s.c. in NCR nude mice and treatments were given as described herein. Tumor size was determined by caliper measurements. (A) EOC tumor proliferation kinetics and (B) terminal tumor mass from mice dosed with vehicle control, paclitaxel, carboplatin, or Compound 1; (C) Excised tumors from representative mice dosed with either vehicle or Compound 1 (50 or 100 444 mg/kg); (D) Tumors from representative mice were lysed and analyzed by western blot analysis for phospho-S6 kinase (p-S6K) and total S6 kinase (S6K). (E) Paraffin-embedded sections of mouse tumors were analyzed for the localization of Endo by IHC. *p=0.02, relative to vehicle.

FIG. 9. Effects of Caspase Inhibitor I (z-VAD-fmk) on Compound 10-induced apoptosis and necrosis in pancreatic adenocarcinoma cells as measure by FACS analysis. A. HPAC, B. MIAPaCa-2. Cells were pre-incubated with 10 μM z-VAD-fmk for 1 h prior to the addition of 10 μM Compound 10. After 42-46 h the cells were harvested and assayed for apoptosis by FACS analysis. As positive caspase-dependent controls HPAC cells were challenged with a Fas-activating antibody (CH-11, Upstate) while MIAPaCa-2 cells were exposed to 50 ng/mL TRAIL (Alexis). In each spectrum, 10,000 gated events were recorded and analysed. All experiments were done in triplicate, error bars represent one standard deviation.

FIG. 10. In Situ caspase activity in pancreatic cancer cells as measured by FACS analysis. (A) A histogram of Caspases-2, -3 and -9 activity in Compound 10 treated (10 μM, 24 hr) and untreated MIAPaCa-2 cells; (B) histogram of caspase-2, -3 and -9 activity in Compound 10 treated (10 μM, 24 hr) and untreated HPAC cells; (C) A histogram of caspase-2 and -3 and -9 activity in Compound 10 treated (10 μM, 24 hr) and untreated PANC-1 cells. All experiments were done in triplicate, error bars represent one standard deviation.

FIG. 11. Western blot analysis of key regulatory proteins integral to apoptosis, and p21 and p53. (A) Western blot analysis of caspase 2, caspase 9, XIAP, Bcl-2, Bid in MIAPaCa-2 cells and Akt in MIAPaCa-2 and HPAC cells. (B) Western blot analysis p2land p53 expression in MIAPaCa-2 and HPAC cells. In both data sets cells were treated with 10 μM Compound 10 for 0, 4, 8, 16, 24 & 48 hrs. Cells were lysed, preparations centrifuged, and lysates collected. Respective cell lysate (25 μg) samples from each time point were separated by SDS-PAGE, transferred to PVDF membranes and probed with antibodies specific to the target antigen. Protein loading was standardised to GAPDH and the GAPDH data shown is representative of replicate studies (RDI). (C) Western blot analysis of cytochrome c release from PANC-1 cells treated with Compound 10 (5 and 10 μg/ml) for 48 hrs. A parallel experiment was performed where the cytoplasmic and mitochondrial fractions were separated before Western blot analyses. Cox-4 was included to show the integrity of the cytoplasmic and mitochondrial preparations and protein loading was standardised to β-actin.

FIG. 12. Effect of Compound 10 on mitochondrial membrane potential of HPAC and MIAPaCa-2 cells. MIAPaCa-2 cells were exposed to. 0 μM (A), or 100 μM (B) Compound 10 for 48 hrs. Aggregated JC-1 red fluorescence from intact, polarized mitochondria is displayed on the Y axis while monomeric JC-1 green fluorescence from apoptotic cells with depolarized mitochondria is displayed on the X axis. The upper left quadrant is defined as polarized cells. The sum of the upper right and lower right quadrants are defined as depolarized cells. In each spectrum, 10,000 gated events were recorded and analysed. Mitochondrial membrane potential was measured in HPAC (C) or MIAPaCa-2 (D) cells over increasing concentrations of Compound 10. All experiments were done in triplicate.

FIG. 13. Effect of Compound 10 on cell cycle progression in MIAPaCa-2, HPAC and PANC-1 cells. (A) Representative FACS histograms of untreated and Compound 10 treated (10 μM, 4 hr and 24 hr) MIAPaCa-2 cells, HPAC cells and PANC-1 cells. Gates: M1=sub-G₀/G₁, M2=G₀/G₁, M3=S, M4=G₂/M and M5=polyploidy cells. In each spectrum, 10,000 gated events were recorded and analysed. All experiments were done in triplicate; (B) Combined plots of cell cycle distribution. Cells were exposed to 10 μM Compound 10 for 0, 4, 24 and 48 hrs. Cells were fixed in ethanol, stained with propidium iodide and analysed by FACS analysis. All experiments were done in triplicate, error bars represent one standard deviation.

### DETAILED DESCRIPTION OF THE INVENTION

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an element” means one element or more than one element.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As used herein the terms “effective amount” and “effective dose” include within their meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” refers to a derivative of the active compound that upon administration to the recipient, is capable of providing directly or indirectly, the parent compound or metabolite, or that exhibits activity itself. Prodrugs are included within the scope of the present invention.

Compounds 1 and 10, exemplified herein, are members of a phenyl-substituted isoflavan family of compounds with anti-proliferative activity against a range of cancer cell lines. The present application describes the activation in human cells by compounds of this family of a caspase-independent non-autophagic cell death pathway. At least in the case of Compound 1, this involves the dephosphorylation of mTOR. As exemplified herein, it is demonstrated that paclitaxel-resistant cell lines undergo caspase-independent cell death in the presence of Compound 1, characterized by down-regulation of p-mTOR, Beclin-1 mitochondrial translocation, nuclear translocation of the nuclease EndoG, and DNA fragmentation. Cell death is not autophagic, proceeding in the presence of apoptotic inducers, and hence is termed herein “caspase-independent apoptosis”. Similarly, Compound 10 is demonstrated to induce cell cycle arrest leading to apoptosis in a range of cell lines. That Compound 10-induced apoptosis proceeds in the presence of a pan-acting caspase inhibitor indicates that both caspase-mediated and caspase-independent pathways of apoptosis are induced.

In one aspect, the present invention provides a method for inducing or promoting apoptosis in a cell, the method comprising exposing to the cell and effective amount of a compound of formula I.

The present invention also provides methods for the treatment or prevention of diseases and disorders associated with reduced or otherwise aberrant apoptosis.

Accordingly, one aspect of the invention provides a method for preventing or treating a disease or disorder in a subject, the method comprising administering to the subject an effective amount of a compound of formula I, wherein the compound induces or promotes apoptosis in at least one cell of the subject. Optionally, the compound is administered in the form of a pharmaceutical composition, which may comprise one or more pharmaceutically acceptable diluents, adjuvants and/or excipients.

It will also be readily appreciated by those skilled in the art that the present invention contemplates the administration of more than one compound of formula I, and/or the administration of at least one compound of formula I in conjunction with at least one additional therapeutic compound or agent.

Compounds useful in the present invention are of the general formula (I):

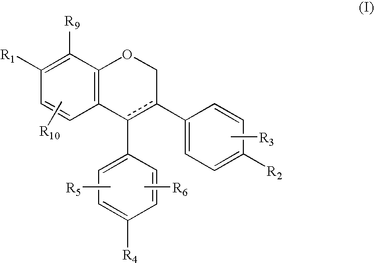

wherein
R₁ is hydrogen, hydroxy, alkyl, alkoxy, halo or OC(O)R₇,
R₂ and R₃ are independently hydrogen, hydroxy, alkoxy, alkyl, cycloalkyl, halo or OC(O)R₇,
R₄, R₅ and R₆ are independently hydrogen, hydroxy, alkoxy, alkyl, cycloalkyl, acyl, amino, C₁₋₄-alkylamino or di(C₁₋₄-alkyl)amino, OC(O)R₇ or OR₈,
R₇ is hydrogen, alkyl, cycloalkyl, aryl, arylalkyl or amino, and
R₈ is aryl or arylalkyl,
R₉ and R₁₀ are independently hydrogen, hydroxy, alkyl, alkoxy or halo, and the drawing [symbol] represents a single bond or a double bond,
   or a pharmaceutically acceptable salt or derivative thereof.

Typically in compounds of formula (I) the substitution pattern of R₂ and R₃ is as shown below:

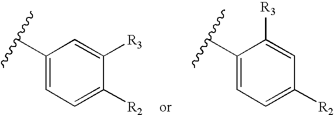

In compounds of formula (I) the substitution pattern of R₄, R₅ and R₆ may be as shown below:

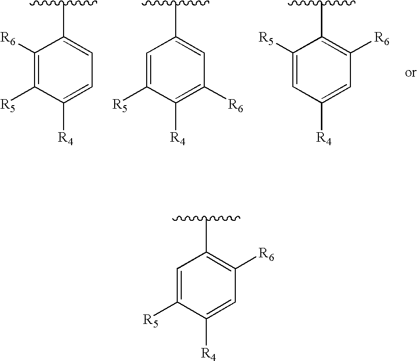

In compounds of formula (I) the drawing [symbol] may represent a single bond.

In an embodiment, in compounds of formula (I):
R₁ is hydroxy, C₁₋₄-alkoxy or OC(O)R₇,
R₂ and R₃ are independently hydrogen, hydroxy, C₁₋₄-alkoxy, halo or OC(O)R₇,
R₄, R₅ and R₆ are independently hydrogen, hydroxy, alkoxy, alkyl, cycloalkyl, acyl, OC(O)R₇, and
R₇ is C₁₋₄-alkyl, phenyl or benzyl,
R₉ is hydrogen, hydroxy, alkyl or halo,
   or a pharmaceutically acceptable salt or derivative thereof.

In an embodiment, in compounds of formula (I):
R₁ is hydroxy, methoxy, ethoxy or acetyloxy,
R₂ and R₃ are independently hydrogen, hydroxy, methoxy, ethoxy, propoxy, isopropoxy, bromo, chloro, fluoro or acetyloxy,
R₄ is hydrogen, hydroxy, methoxy, ethoxy, propoxy, isopropoxy or acetyloxy, and
R₅ and R₆ are independently hydrogen, hydroxy, methoxy, ethoxy, propoxy, isopropoxy, acetyl, or acetyloxy,
R₉ is hydrogen, hydroxy, methyl, methoxy, bromo, chloro, fluoro or acetyloxy,
R₁₀ is hydrogen,
   or a pharmaceutically acceptable salt or derivative thereof.

Compounds of formula (I) may have the following substituents where:
R₁ is hydroxy, methoxy or acetyloxy,
R₂ and R₃ are independently hydrogen, hydroxy, methoxy, bromo or acetyloxy,
R₄ and R₆ are independently hydrogen, hydroxy, methoxy or acetyloxy,
R₅ and R₁₀ are hydrogen, and
R₉ is hydrogen, methyl or bromo,
   or a pharmaceutically acceptable salt or derivative thereof.

In an embodiment, R₉ is methyl.

According to embodiments of the invention compounds of formula (I) include:
3-(4-hydroxyphenyl)-4-(4-methoxyphenyl)-8-methylchroman-7-ol (Compound 1);
3-(4-methoxyphenyl)-4-(4-methoxyphenyl)-7-methoxy-8-methylchroman (Compound 2);
3-(3,4-dimethoxyphenyl)-4-(4-methoxyphenyl)-8-methylchroman-7-ol (Compound 3);
3-(4-methoxyphenyl)-4-(4-methoxyphenyl)-8-methylchroman-7-ol (Compound 4);
3-(4-hydroxyphenyl)-4-(4-methoxyphenyl)-7-methoxy-8-methylchroman (Compound 5);
3-(3-methoxyphenyl)-4-(4-methoxyphenyl)-8-methylchroman-7-ol (Compound 6);
3-(3,4-dihydroxyphenyl)-4-(4-methoxyphenyl)-7-methoxy-8-methylchroman (Compound 7);
3-(3-hydroxyphenyl)-4-(4-methoxyphenyl)-8-methylchroman-7-ol (Compound 8);
3-(3,4-dihydroxyphenyl)-4-(4-methoxyphenyl)-8-methylchroman-7-ol (Compound 9);
   or a pharmaceutically acceptable salt thereof.

In another embodiment, R₉ is hydrogen.

According to additional embodiments compounds of formula (I) include:
3-(4-hydroxyphenyl)-4-(4-methoxyphenyl)chroman-7-ol (Compound 10);
3-(4-hydroxyphenyl)-4-phenylchroman-7-ol (Compound 11);
3-(4-hydroxyphenyl)-4-(3-methoxyphenyl)chroman-7-ol (Compound 12);
3-(3,4-dimethoxyphenyl)-4-(4-methoxyphenyl)chroman-7-ol (Compound 13);
3-(4-hydroxyphenyl)-4-(4-methylphenyl)chroman-7-ol (Compound 14);
3-(4-methoxyphenyl)-4-(4-methoxyphenyl)-7-methoxychroman (Compound 15);
3-(4-hydroxyphenyl)-4-(2,6-dimethoxy-4-hydroxyphenyl)chroman-7-ol (Compound 16);
3-(4-hydroxyphenyl)-4-(2-hydroxyphenyl)chroman-7-ol (Compound 17);
3-(4-hydroxyphenyl)-4-(3-acyl-2-hydroxy-4-methoxyphenyl)chroman-7-ol (Compound 18);
3-(3-hydroxyphenyl)-4-(3-methoxyphenyl)chroman-7-ol (Compound 19);
3-(4-hydroxyphenyl)-4-(4-hydroxyphenyl)chroman-7-ol (Compound 20);
3-(4-bromophenyl)-4-(4-methoxyphenyl)chroman-7-ol (Compound 21);
3-(4-hydroxyphenyl)-4-(3-methoxyphenyl)chroman-7-ol (Compound 22);
3-(4-hydroxyphenyl)-4-(3-aminophenyl)chroman-7-ol (Compound 23);
3-(4-hydroxyphenyl)-4-(4-phenoxyphenyl)chroman-7-ol (Compound 24);
   or a pharmaceutically acceptable salt thereof.

The compounds of formula (I) according to the invention include two chiral centres. The present invention includes all the enantiomers and diastereoisomers as well as mixtures thereof in any proportions. The invention also extends to isolated enantiomers or pairs of enantiomers. Methods of separating enantiomers and diastereoisomers are well known to person skilled in the art.

It will be clear to persons skilled in the art that in the compounds of formula (I) the aryl substituents on the heterocyclic ring can be cis or trans relative to each other.

Compounds to which particular embodiments of the present invention relate are:

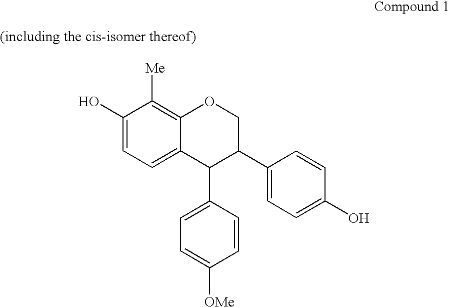

or a pharmaceutically acceptable salt thereof; and

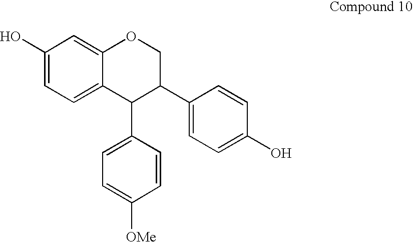

or a pharmaceutically acceptable salt thereof.

The term “alkyl” is taken to include straight chain and branched chain saturated alkyl groups of 1 to 6 carbon atoms, such as methyl, ethyl, propyl, isopropyl, butyl, isobutyl, secbutyl, tertiary butyl, pentyl and the like. The alkyl group more preferably contains preferably from 1 to 4 carbon atoms, especially methyl, ethyl, propyl or isopropyl.

Cycloalkyl includes C₃₋₆ cycloalkyl such as cyclopropyl, cyclobutyl, cyclopentyl and cyclohexyl.

The alkyl group or cycloalkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl. The alkyl group may not bear any substituents.

The term “aryl” is taken to include phenyl, benzyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy, nitro or halo.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The compounds of the invention include all salts, such as acid addition salts, anionic salts and zwitterionic salts, and in particular include pharmaceutically acceptable salts as would be known to those skilled in the art. Pharmaceutically acceptable salts include those formed from: acetic, ascorbic, aspartic, benzoic, benzenesulphonic, citric, cinnamic, ethanesulphonic, fumaric, glutamic, glutaric, gluconic, hydrochloric, hydrobromic, lactic, maleic, malic, methanesulphonic, naphthoic, hydroxynaphthoic, naphthalenesulphonic, naphthalenedisulphonic, naphthaleneacrylic, oleic, oxalic, oxaloacetic, phosphoric, pyruvic, p-toluenesulphonic, tartaric, trifluoroacetic, triphenylacetic, tricarballylic, salicylic, sulphuric, sulphamic, sulphanilic and succinic acid.

Pharmaceutically acceptable derivatives include solvates, pharmaceutically active esters, prodrugs or the like. This also includes derivatives with physiologically cleavable leaving groups that can be cleaved in vivo to provide the compounds of the invention or their active moiety. The leaving groups may include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted compounds of the invention are readily cleavable to the corresponding hydroxy substituted compounds.

Compounds of formula I to which the present invention relates are believed to have favourable toxicity profiles with normal cells and good bioavailability. These compounds are described in International Patent Applications PCT/AU2005/001435 (published as WO 2006/032085) and PCT/AU2005/001436 (published as WO 2006/032086), the disclosures of which are incorporated herein by reference.

mTOR (mammalian Target of Rapamycin) is a serine/threonine protein kinase involved in the regulation of cell growth and proliferation, cell survival, cell motility and protein synthesis (see Hay and Sonenberg, 2004). An important function of mTOR is the control of translation via regulation of S6k and 4EBP1. Activation of the pathway results in enhanced translation, enhanced cell mass, and cell cycle progression. mTOR is also an essential part of tumor progression capable of integrating proliferative, antiapoptotic, and angiogenic signalling. There is increasing interest in the therapeutic application of mTOR inhibitors, exemplified by sirolimus and more recently the sirolimus analogs temsirolimus and everolimus, for treating a variety of diseases and conditions, including restenosis, transplant rejection and rheumatoid arthritis.

Embodiments of the present invention find particular application in the therapeutic or prophylactic treatment of diseases and conditions which are associated with aberrant or otherwise unwanted cell growth and/or proliferation and diseases and conditions in which the inhibition of mTOR activity is beneficial. As used herein the term “associated with” as used with reference to diseases and conditions associated with aberrant or otherwise unwanted cell growth and/or proliferation, means that a disease or condition may be caused by, may cause, or may otherwise be associated with abnormal cellular proliferation. The abnormal cellular proliferation may be in any cell type, including for example a myocardial or immune cell (typically a proliferating or abnormally proliferating T cell). Preferably the cell is not a cancer cell.

Thus, suitable diseases and conditions to which embodiments of the invention find applicability include, but are not limited to, stenosis, restenosis, transplant rejection and rheumatoid arthritis and diseases and conditions associated with abnormal immune cell proliferation or activation. By way of example, the isoflavonoid compounds may be administered in the period immediately prior to and following vascular intervention such as coronary or vascular angioplasty as a means to reduce or eliminate the abnormal proliferative response that currently leads to clinically significant restenosis. The transplant rejection may be of any tissue or organ. Those skilled in the art will appreciate that a wide range of diseases and conditions are associated with aberrant or otherwise unwanted cell growth and/or proliferation and the present invention finds applicability in the treatment of any such disease or condition.

Embodiments of the present invention find application in the inhibition of immune cell proliferation by the induction of apoptosis and thus in the therapeutic and prophylactic treatment of diseases or conditions associated with abnormal immune cell, particularly T cell, proliferation or stimulation. Typically, in the context of the present invention, abnormal T cell proliferation means abnormally rapid proliferation. Diseases and conditions include, by way of non-limiting example, T cell leukemias, autoimmune diseases, and transplant or graft rejections such as graft versus host disease. Autoimmune diseases include, but are not limited to, cirrhosis, psoriasis, lupus, rheumatoid arthritis, Addison's disease, infectious mononucleosis, Sézary's syndrome and Epstein-Barr virus infection. Those skilled in the art will appreciate that a wide range of diseases and conditions are associated with abnormal T cell proliferation and the present invention finds applicability in the treatment of any such disease or condition.

Compounds as described herein may be administered alone or in conjunction with other active agents for treating diseases and conditions associated with aberrant or otherwise unwanted cell growth and/or proliferation and diseases and conditions in which the inhibition of mTOR activity is beneficial. By way of example, for the treatment of restenosis, compounds of the invention may be administered together with other mTOR inhibitors such as sirolimus, temsirolimus or everolimus. Where such combination therapy is to be administered the active agents may be administered sequentially or simultaneously.

Also provided herein are methods for augmenting existing treatment regimes for subjects suffering from diseases or conditions that are associated with aberrant or otherwise unwanted cell growth and/or proliferation, the methods comprising administering to subjects in need thereof an effective amount of a compound of formula I as described herein or a pharmaceutically acceptable salt or prodrug thereof. Thus, for example, it is contemplated that compounds as described herein may be used in conjunction with existing therapeutic treatments for a range of diseases and conditions where a reduction in proliferating T cell activity and/or proliferation would be of benefit. That is, the administration of an immunomodulating effective amount of a compound described herein may improve the ability of a patient to respond to an existing treatment for the disease or condition suffered by the patient.

By “immunomodulating effective amount” is meant an amount or dose of the compound that is sufficient to modulate the immune system or immune response as desired. This immunomodulating amount may be a subtherapeutic dose of the compound, where a therapeutic dose is a dose that is sufficient to have a non-immunomodulatory, therapeutic effect against a particular disease or condition. That is, a smaller amount or dose of the compound may be administered to achieve an immunomodulatory effect than the amount or dose required to be administered to achieve a non-immunomodulatory therapeutic effect.

According to the methods of present invention isoflavonoid compounds and compositions comprising such isoflavonoids may be administered by any suitable route, either systemically, regionally or locally. The particular route of administration to be used in any given circumstance will depend on a number of factors, including the nature of the condition to be treated, the severity and extent of the condition, the required dosage of the particular compound to be delivered and the potential side-effects of the compound. For example, in circumstances where it is required that appropriate concentrations of the desired compound are delivered directly to the site in the body to be treated, administration may be regional rather than systemic. Regional administration provides the capability of delivering very high local concentrations of the desired compound to the required site and thus is suitable for achieving the desired therapeutic or preventative effect whilst avoiding exposure of other organs of the body to the compound and thereby potentially reducing side effects.

By way of example, administration according to embodiments of the invention may be achieved by any standard routes, including intracavitary, intravesical, intramuscular, intraarterial, intravenous, intraocular, subcutaneous, topical or oral.

According to particular embodiments of the present invention isoflavonoid compounds and compositions comprising such isoflavonoids may be incorporated into implantable medical devices for administration. Stents and catheters in particular are adapted to be implanted into a patient's body lumen, such as a blood vessel e.g. coronary artery, bile ducts, oesophagus, colon, trachea or large bronchi, ureters and urethra. Stents are particularly useful in the treatment of atherosclerotic stenosis and aneurysms.

Stents are typically implanted within a vessel or lumen in a contracted state, and can be expanded when in place in the vessel in order to maintain the patency of the vessel to allow fluid flow through the vessel. Stents have a support structure such as a metallic structure to provide the strength required and are often provided with an exterior surface coating to provide a biocompatible and/or hemocompatible surface. The coating is typically a polymeric material and may be loaded with therapeutically active agents for release at a specific intravascular site for action on the surrounding vessel or downstream thereof.

Drug-eluting stents have shown great promise in treating coronary artery disease, specifically in terms of reopening and restoring blood flow in arteries stenosed by atherosclerosis. Restenosis rates after using drug-eluting stents during percutaneous intervention are significantly lower compared to bare metal stenting and balloon angioplasty.

As will be appreciated by those having ordinary skill in the art, a drug-eluting stent used in accordance with the present invention can be virtually of any type. The drug-eluting stent may be formed at least in part of a medical grade metallic material such as stainless steel, platinum, titanium, tantalum, nickel-titanium, cobalt-chromium, and alloys thereof. The stents may also be made of bioabsorbable material. Typically these stents are fully resorbable structures having metal-like scaffolding, generally use standard balloon deployment, and can be loaded with drug. Examples of the materials with which bioabsorbable stents can be made is polylactic acid (PLA), a biodegradable, thermoplastic, aliphatic polyester, or polylactic-co-glycolic acid (PLGA), a biodegradable biocompatible copolymer or magnesium.

The active agent can be attached to the implantable medical device surface by any means that provides a drug-releasing platform. Binding can be achieved covalently, ionically, or through other molecular interactions including hydrogen bonding and van der Waals forces. Coating methods include, but are not limited to precipitation, coacervation, and crystallization. More typically, the active agent is complexed with a suitable biocompatible polymer. The polymer-drug complex is then used to either form a controlled-release medical device, integrated into a preformed medical device or used to coat a medical device as would be well known to a person skilled in the art. The coatings can be applied as a liquid polymer/solvent matrix. The liquid coating can be applied by pad printing, inkjet printing, rolling, painting, spraying, micro-spraying, dipping, wiping, electrostatic deposition, vapour deposition, epitaxial growth, combinations thereof and other methods of achieving controlled drug release with the active agents are contemplated as being part of the present invention.

Examples of suitable polymers for use in coating the implantable medical devices include poly(ethylene-co-vinyl alcohol) (EVAL), poly(N-vinylpyrrolidone) (PVP), ethyl cellulose, cellulose acetate, carboxymethyl cellulose, cellulosics, chitin, chitosan, poly(vinyl alcohol), heparin, dextran, dextrin, dextran sulfate, collagen, gelatin, hyaluronic acid, chondroitan sulfate, glycosaminoglycans, poly[(2-hydroxyethyl)methylmethacrylate], polyurethanes, poly(ether urethanes), poly(ester urethanes), poly(carbonate urethanes), thermoplastic polyesters, solvent soluble nylons, poly(acrylamide), poly(acrylic acid), copolymers of acrylic acid and acrylates, poly(methacrylic acid), copolymers of methacrylic acid and methacrylates, and blends thereof.

Persons skilled in the art will appreciate that different polymers are better suited to different solvents in the formation of the medical device coatings. Examples of suitable solvents include acetone, ethyl acetate, chloroform, dichloromethane, DMAC, DMSO, DMF, THF, formamide, N-methyl-2-pyrrolidone (NMP), sulfolane, benzyl alcohol, cyclohexanol, phenol, formic acid, m-cresol, p-cresol, trifluoroacetic acid, glycerol, ethylene glycol, propylene glycol, ethanol, propanols and mixtures thereof. The active agent must also be suitably soluble or dispersible in the polymer/solvent mixture and retain its activity during the coating process. The polymer may be adhered to the stent using conventional metal-polymer adhesion techniques, such as by dipping, spraying, wiping, and brushing, which are known in the art. These processes may be followed by web clearing operations that can include blowing air or spinning and drying operations including evaporation, heating and subjecting the coating to reduced pressure to remove the solvent and set the drug containing polymer. The polymer coating can have a thickness in the range of about 1 micron to about 10 microns or more and more that one coating may be desired including primer coatings and protective layer coatings.

The active agent loading is typically between about 0.1 and about 10 mass % of the total mass of the formulation used to make the drug-polymer layer. The drug can include additional substances capable of exerting a therapeutic or prophylactic effect for a patient or for use in increasing drug delivery, as a preservative, stabiliser or the like.

Therapeutic drugs suitable for co-application on stents include, but are not limited to, antiproliferatives including paclitaxel and rapamyacin, antithrombins, immunosuppressants including sirolimus, antilipid agents, anti-inflammatory agents, antineoplastics, antiplatelets, angiogenic agents, anti-angiogenic agents, vitamins, antimitotics, metalloproteinase inhibitors, NO donors, estradiols, anti-sclerosing agents, and vasoactive agents, endothelial growth factors, estrogen, beta blockers, AZ blockers, hormones, statins, insulin growth factors, antioxidants, membrane stabilizing agents, calcium antagonists, retenoid, bivalirudin, phenoxodiol, etoposide, ticlopidine, dipyridamole and trapidil alone or in combinations with any therapeutic agent mentioned herein. Therapeutic agents also include peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense, oligonucleotides, monoclonal antibodies, platelets, prions, viruses, bacteria, and eukaryotic cells such as endothelial cells, stem cells, ACE inhibitors, monocyte/macrophages or vascular smooth muscle cells to name but a few examples. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the therapeutic layer. Therapeutic agents may also be radioactive isotopes or agents activated by some other form of energy such as light or ultrasonic energy, or by other circulating molecules that can be systemically administered. Therapeutic agents may perform multiple functions including modulating angiogenesis, restenosis, cell proliferation, thrombosis, platelet aggregation, clotting and vasodilation. Anti-inflammatories include non-steroidal anti-inflammatories (NSAID), such as aryl acetic acid derivatives, e.g., Diclofenac; aryl propionic acid derivatives, e.g., Naproxen; and salicylic acid derivatives, e.g., aspirin and Diflunisal. Anti-inflammatories also include glucocoriticoids (steroids) such as dexamethasone, prednisolone and triamcinolone. Anti-inflammatories may be used in combination with antiproliferatives to mitigate the reaction of the tissue to the antiproliferative.

Drug-eluting stents and catheters as described herein may be utilised in any part of the vasculature including neurological, carotid, coronary, renal, aortic, iliac, femoral or other peripheral vasculature. The stents can deliver the active agents to the site of implantation or downstream of the site.

The drug-eluting stents can have multiple layers created independently and as such individual chemical compositions and pharmacokinetic properties can be imparted to each layer. Each of the layers may include one or more agents in the same or different proportions from layer to layer. Changes in the agent concentration between layers can be used to achieve a desired delivery profile. For example, a decreasing release of drug for about 24 hours can be achieved. In another example, an initial burst followed by a constant release for about one week can be achieved. Other examples can deliver an agent over a sustained period of time, such as several days to several months. Substantially constant release rates over time period from a few hours to months can be achieved. The layers may be solid, porous, or filled with other drugs or excipients and the like.

In employing methods of the invention, isoflavonoid compounds may be formulated in pharmaceutical compositions. Suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof. The diluent, adjuvant or excipient may be a solid or a liquid, or both, and may be formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

Examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions.

Formulations suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration typically conveniently comprise sterile aqueous preparations of the active compounds, which preparations may be isotonic with the blood of the intended recipient. These preparations are typically administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound(s) and are administered at a rate of 0.1 ml/minute/kg or as appropriate.

Formulations for infusion, for example, may be prepared employing saline as the carrier and a solubilising agent such as a cyclodextrin or derivative thereof. Suitable cyclodextrins include α-cyclodextrin, β-cyclodextrin, γ-cyclodextrin, dimethyl-β-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. More preferably the cyclodextrin is hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Formulations suitable for rectal administration are typically presented as unit dose suppositories. These may be prepared by admixing the active compound with one or more conventional solid carriers, for example, cocoa butter, and then shaping the resulting mixture.

Formulations or compositions suitable for topical administration to the skin may take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for inhalation may be delivered as a spray composition in the form of a solution, suspension or emulsion. The inhalation spray composition may further comprise a pharmaceutically acceptable propellant such as carbon dioxide or nitrous oxide or a hydrogen containing fluorocarbon such as 1,1,1,2-tetrafluoroethane, 1,1,1,2,3,3,3-heptafluoro-n-propane or mixtures thereof.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. Formulations suitable for transdermal administration may also be delivered by iontophoresis (see, for example, Pharmaceutical Research 3 (6), 318 (1986)) and typically take the form of an optionally buffered aqueous solution of the active compound. For example, suitable formulations may comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

According to the present invention, compounds and compositions may be administered either therapeutically or preventively. In a therapeutic application, compounds and compositions are administered to a patient already suffering from a disease or disorder or experiencing symptoms, in an amount sufficient to cure or at least partially arrest the disease or disorder, symptoms and/or any associated complications. The compound or composition should provide a quantity of the active compound sufficient to effectively treat the patient.

The effective dose level of the administered compound for any particular subject will depend upon a variety of factors including: the type of condition being treated and the stage of the condition; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis. By way of example only, an effective dosage may be expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; or about 1.0 mg to about 250 mg per kg body weight per 24 hours. More typically, an effective dose range is expected to be in the range of about 10 mg to about 200 mg per kg body weight per 24 hours.

Further, it will be apparent to those of ordinary skill in the art that the optimal quantity and spacing of individual dosages will principally be determined by the nature and extent of the condition being treated, the form, route and site of administration, and the individual being treated. Suitable conditions can be determined by conventional techniques.

It will also be apparent to those of ordinary skill in the art that the optimal course of treatment, such as, the number of doses of the composition given per day for a defined number of days, can be ascertained by those skilled in the art using conventional course of treatment determination tests.

In accordance with the methods of the invention, isoflavonoid compounds or pharmaceutically acceptable derivatives prodrugs or salts thereof can be co-administered with other active agents that do not impair the desired action, or with agents that supplement the desired action, such as antibiotics, antifungals, antiinflammatories, lipid lowering agents, platelet aggregation inhibitors, antithrombotic agents, calcium channel blockers, corticosteroids or antiviral compounds. The particular agent(s) used will depend on a number of factors and will typically be tailored to the disease or disorder to be treated. The co-administration of agents may be simultaneous or sequential. Simultaneous administration may be effected by the compounds being formulated in a single composition, or in separate compositions administered at the same or similar time. Sequential administration may be in any order as required.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### Examples

### General Methods

### Compound 1 Studies:

Cell lines and culture conditions. Established human EOC cell lines, A2780 and A2780/CP70 (gifts from Dr. T C Hamilton) (Behrens et al, 1997) were propagated in RPMI plus 10% fetal bovine serum (Gemini Bio-Products, Woodland, Calif.). Primary EOC cell lines were isolated from malignant ovarian ascites or explanted from ovarian tumors and cultured as previously described (Alvero et al, 2006). Purity of the EOC cells was 100% as determined by immuno-staining for cytokeratin 7 antigen. All cell lines were grown at 37° C. in a 5% CO₂ atmosphere.

Cell viability assay. Cell viability was determined as previously reported (Alvero et al., 2006). Briefly, cells (5×10³) were plated in triplicate wells in a 100 μml volume per well of a 96-well microtiter plate (BD Biosciences/Pharmingen, San Diego, Calif.). The cells were grown to 70% confluence and then incubated in reduced-serum phenol-depleted Opti-MEM medium (Invitrogen-GIBCO, Carlsbad, Calif.) for 4 h prior to treatment. Compound 1 (Novogen, Inc., NSW, Australia) was added to the medium from 10 mg/ml stock to give various final concentrations as described in the results section. Following 24 h treatment, cell viability was evaluated using the CellTiter 96® AQueous One Solution Cell Proliferation Assay (Promega Corporation, Madison, Wis.) according to the manufacturer's instructions. Optical densities of the samples were measured at 490 nm using an automatic microplate reader (Model 550, Bio-Rad, Hurcules, Calif.). The values from the treated cells were compared with the values generated from the untreated control and reported as percent viability. Each experiment was done in triplicate.

For experiments using the caspase inhibitor, Z-VAD-FMK (Sigma Aldrich, St. Louis, Mo.), the inhibitor was added to the cultures 30 mins prior to treatment to yield a final concentration of 20 μM. For experiments using the autophagy inhibitor, 3-methyladenine (Sigma Aldrich), the inhibitor was added 1 h prior to treatment to yield final concentration of 10 mM.

Flow cytometry with Hoechst and Propidium iodide staining. After treatment, cells were trypsinized, washed twice with PBS, and resuspended at 1×10⁶ cells/ml. Cells were then stained with 5 μg/ml Hoechst 33342 (Invitrogen-Molecular Probes, Carlsbad, Calif.) and 1 μg/ml Propidium iodide (Sigma Aldrich) and incubated for 15 mins in the dark. Afterwards, data was acquired using BD LSR II System and analyzed using FloJo FACS analysis software (Tree Star, Inc., Ashland, Oreg.).

Protein preparation and cellular fractionation. After drug treatment, protein was extracted and measured as previously described (Alvero et al, 2006). For separation of the cytoplasmic and mitochondrial fractions, cell pellets were processed using the ApoAlert™ Cell Fractionation Kit (BD Biosciences, San Jose, Calif.) according to the manufacturer's instructions. For separation of the cytoplasmic and nuclear fractions, pellets were processed using NE-PER Nuclear and Cytoplasmic Extraction (Pierce Biotechnology, Inc., Rockford, Ill.).

Caspase-3/7, -8, and -9 activity assay. Ten μg of protein in a 50 μl total volume was mixed with 50 μl of equilibrated Caspase-Glo¹⁰ 3/7, 8, or 9 reagents (Promega). After incubating at room temperature for 1 h, luminescence was measured using TD 20/20 Luminometer (Turner Designs, Sunnyvale, Calif.). Blank values were subtracted and fold-increase in activity was determined in relation to the control non-treatment.

SDS-PAGE and Western blots. Cell lysates (20 μg protein) were denatured in sample buffer (2.5% SDS, 10% glycerol, 5% β-mercapto-ethanol, 0.15 M Tris (pH=6.8) and 0.01% bromophenol blue) and subjected to 12% SDS-PAGE as previously described (Kamsteeg et al, 2003). The following antibodies and concentrations were used: mouse anti-XIAP (BD, 1:1,000), rabbit anti-MAP LC3 (1:200), rabbit anti-actin (Sigma, 1:10,000), rabbit anti-phosphorylated Akt (Cell Signaling, 1:1,000), rabbit anti-Akt (Cell Signaling, 1:1000), rabbit anti-phosphorylated mTOR (Cell Signaling, 1:1000), rabbit anti-mTOR (Cell Signaling, 1:1000), rabbit anti-cytochrome c (Clontech Laboratories, Inc, Mountainview, Calif., 1:100), rabbit anti-Omi (R&D Systems, Minneapolis, Minn., 1:5000), rabbit anti-Bid (Cell Signaling, 1:1000), mouse anti-Bax (BD Pharmingen, 1:250), rabbit anti-VDAC (Sigma Aldrich, 1:2000), mouse anti-Bcl2 (BD Pharmingen, 1:500), rabbit anti-AIF (Sigma Aldrich, 1:1000), rabbit anti-EndoG (Sigma Aldrich, 1:1000), mouse anti-topoisomerase I (BD Pharmingen, 1:10,000). Proteins were visualized using enhanced chemiluminescence (Pierce, Rockford Ill.).

Analysis of phoshpo-proteins using xMAP technology. After treatment, cells were lysed and lysates were used to measure the phosphorylation status of 8 proteins using the Beadlyte® 8-plex Multi-Pathway Signaling kit (Millipore, Billerica, Mass.) according to manufacturer's instructions. Data was acquired using the BioPlex System (BioRad, Hercules, Calif.).

Assay of mitochondrial depolarization using JC-1. After treatment, cells were trypsinized and stained with JC-1 dye using the Mitocapture™ mitochondrial apoptosis detection kit (BioVision Research Products, Mountain View, Calif.) according to manufacturer's instruction. Data was acquired using BD LSR II System and analyzed using BD FACSDiva Software™.

Immunoprecipitation. Beclin-1 was immunoprecipitated from the mitochondrial fraction of cells treated for 1 h with 10 μg/ml Compound 1 using the Catch and Release® v2.0 Reversible Immunoprecipitation System (Millipore, Billerica, Mass.) and anti-rabbit Beclin-1 (Abcam, Cambridge, Mass.) according to manufacturer's instruction.

Mouse xenograft studies. The in vivo study as approved by the Yale University Institutional Animal Care & Use Committee. Cells (1×10⁶ cells) were resuspended in 200 μ1 total volume of 50% RPMI and 50% BD Matrigel Matrix (BD Biosciences, Bedford, Mass.) and injected subcutaneously into the right flank of NCR nude mice. Intra-peritoneal therapy commenced eight days post-inoculation as follows: Paclitaxel 10 mg/kg q×3d, Carboplatin 40 mg/kg q×7d, and Compound 1 in 20% HPBCD, 100 mg/kg every day. Control groups received 20% HPBCD in PBS. Mice were treated and observed for 3 weeks. Tumor size was determined by caliper measurements and anti-tumor activity was analyzed with respect to maximal tumor inhibition (treated/control, T/C) as described previously (Kamsteeg et al, 2003).

Immunohistochemistry. Immunohistochemistry was performed as described previously (Kelly et al, 2006) using rabbit anti-EndoG (Lifespan Biosciences, Seattle, Wash.) at 1:100 dilution.

### Compound 10 Studies:

Cells. Human pancreatic cell lines HPAC, MIAPaCa-2 and PANC-1 were obtained from the ATCC. (Manassas, Va.). The cells were maintained in DMEM media with Gln and high glucose (Mediatech, Manassas, Va.) with 10% fetal bovine serum (FBS, HyClone, Logan, Utah) and pencillin/streptomycin.

Antibodies. Antibodies utilised in this study specific to p53 and, p21 were obtained from (Calbiochem, EMD, San Diego, Calif.), Caspase 9, XIAP, COX IV, Bcl2 and Akt (Cell Signaling Technology, Danvers, Mass.), Caspase 2, Cytochrome c and Bid (BD Pharmingen, San Diego, Calif.). GAPDH (RDI, Concord, Mass.) or β-actin (Sigma, St. Louis, Mo.) was used as a housekeeping gene to standardize the protein loading on the gels and blots.

Cells Viability. Cell viability was measured on 96 well plates using a MTS assay (CellTiter 96 Aqueous One, Promega, Madison, Wis.) as described previously (Alvero et al., 2006).

FACS Analysis. Apoptosis was measured by FACS analysis using Annexin-V-FITC and propidium iodide (PI) (Biovision, Mountain View, Calif.) as probes (Ahn et al., 2003). Mitochondrial membrane potential was measured by FACS analysis using JC-1 (Biovision) as a probe (Ahn et al., 2003). Cell cycle was analyzed on ethanol-fixed, RNaseA-treated cells staining with PI. In each spectrum, 10,000 gated events were recorded and analyzed with a FACSCalibur (BD Biosciences. San Jose, Calif.). All experiments were done in triplicate, error bars represent one standard deviation. Caspase inhibitor I (zVAD-fmk) was obtained from Calbiochem.

Western blot analysis. Cells were seeded on 100 mm tissue culture plates and grown overnight to ˜90% confluence. Cells were then treated with the drug combinations for indicated time points. After the final treatment step, the cells were lysed with 500 μL of a lysis buffer containing 1% Nonidet P-40, 0.1% SDS, 0.5% deoxycholate, 150 mM NaCl, 50 mM Tris-HCl pH7.5, 1 mM EDTA, 1 mM PMSF and protease inhibitors (Roche) on ice for 30 minutes. Cell lysates were harvested with a sterile cell scraper and centrifuged at 15,000×g for 15 minutes to remove cell debris. Protein was assayed by the BCA method (Pierce) and 50 μg aliquots were separated on 12%, T, 2.6%C SDS-PAGE gels. Proteins were electrophoretically transferred to PVDF membranes (Immobilon P, Millipore, Billerica, Mass.) by the wet transfer method. These membranes were probed with specific antibodies and with anti-GAPDH which was used as the housekeeping gene to standardize the protein loading. The blots were developed on X-ray films using HRP-conjugated secondary antibodies (Southern Biotech, Birmingham, Ala.) and a chemiluminescent substrate (ECL, Amersham, N.Y.).

Caspase Activity After 48 h treatment with 10 μM Compound 10, HPAC, MIAPaCa-2 and PANC-1 cells were harvested by trypsinization and resuspended in DMEM media with 10% FBS. The cells were incubated with caspase substrates for 1 h at 37° C., 5% CO2, then probed with Annexin-V-Cy5 and sytox blue. Caspase +ve cells were gated as shown in the histograms and these cells are indicated on the Annexin-V-Cy5/sytox blue spectra as blue cells. In situ caspase activity was assayed using fluorescent substrates. For Caspase 2, FITC-VDVAD-fmk (Biovision K182-100), for Caspase 3, FITC-DEVD-fmk (K183-100) and for Caspase 9, FITC-LEHD-fmk (K199-100) was used. These substrates bind irreversibly to their respective activated caspases, and caspase activation was quantitated by flow cytometry. After 1 hr incubation at 37° C. in 5% CO₂, the cells were washed with DPBS containing 5% FBS and resuspended in Annexin-V binding buffer containing Annexin-V-Cy5 (Biovision K103-3) and sytox blue (Invitrogen), a dye that binds necrotic cells. Caspase activation, apoptosis and necrosis were measured simultaneously using a violet, blue and red laser in a BD LSRII flow cytometer.

### Example 1

### Compound 1-Induced Cell Death Involves Caspase-Independent DNA Fragmentation

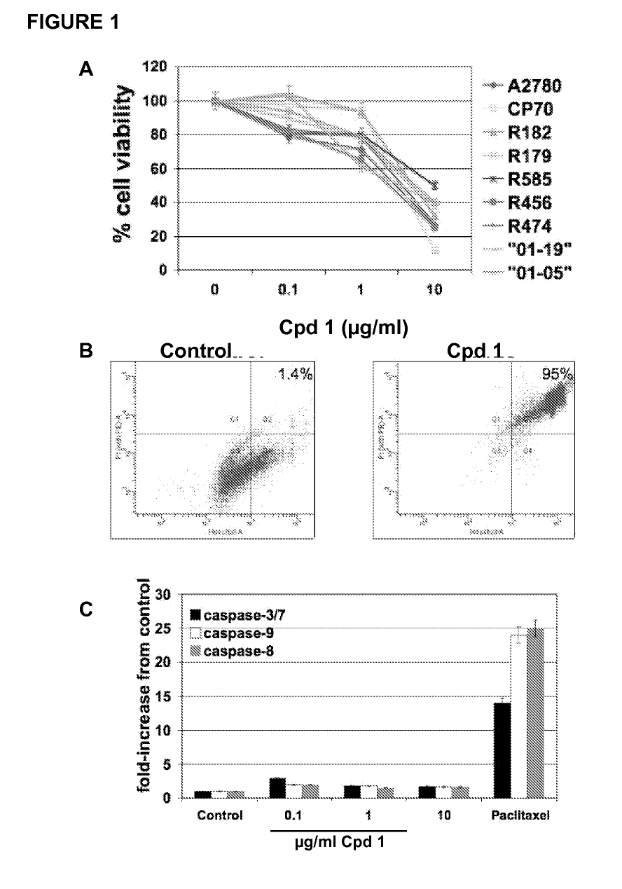

Compound 1 has been found to decrease cell viability in a range of cancer cells including some EOC cell lines (data not shown). Therefore, the inventors' first objective was to determine the effect of Compound 1 on a panel of primary cultures of EOC cells isolated from either ascites or tumor tissue, which include cultures that are paclitaxel-resistant (R182, R456) (Kelly et al, 2006). These cultures express high levels of the anti-apoptotic proteins XIAP and FLIP (16). Paclitaxel-sensitive, as well as paclitaxel-resistant cultures showed a significant reduction in the percentage of viable cells after treatment with Compound 1 with GI₅₀ between 5 and 10 μg/ml (FIG. 1A).

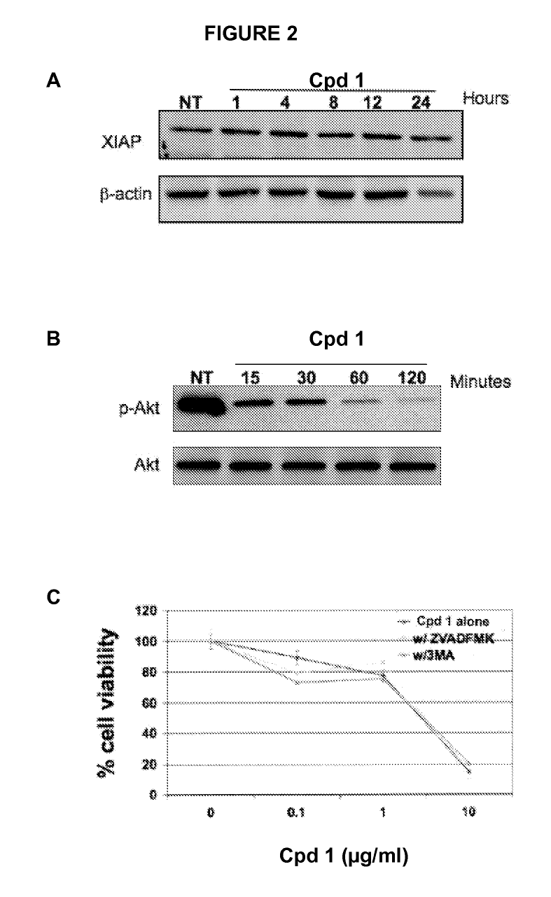

To determine if the decrease in cell viability was due to apoptosis, cells were stained with Hoechst and Propidium Iodide (PI) and analyzed by flow cytometry. In addition, the activity of caspases-3/7, -8, and -9 was measured and the status of two anti-apoptotic molecules, XIAP and phosphorylated Akt (p-Akt) determined. Compound 1 induced a significant increase in the incidence of fragmented DNA with 95% of cells staining positively for both Hoechst and PI after 24 h (FIG. 1B). However, in contrast to the increase in caspase activity observed after treatment with the known apoptotic inducer, Paclitaxel, no changes in caspase-3/7, -8, and -9 activities were observed after treatment with Compound 1 (FIG. 1C). In addition, no change in the status XIAP (an anti-apoptotic protein) was seen (FIG. 2A). Interestingly, however, a strong down-regulation of p-Akt was observed as early as 15 mins post-Compound 1 treatment (FIG. 2B).

DNA fragmentation in the absence of caspase activation suggests the involvement of a caspase-independent pathway. To conclusively show that Compound 1-induced cell death is caspase-independent, cells were treated with increasing concentrations of Compound 1 in the presence or absence of the pan-caspase inhibitor Z-VAD-FMK. Inhibition of caspases had no impact on Compound 1-induced cell death (FIG. 2C). Taken together, these results suggest that Compound 1-induced cell death proceeds via a caspase-independent-, but possibly p-Akt dependent-pathway culminating in DNA fragmentation.

### Example 2

### Autophagy is Not Required for Cell Death Induced by Compound 1

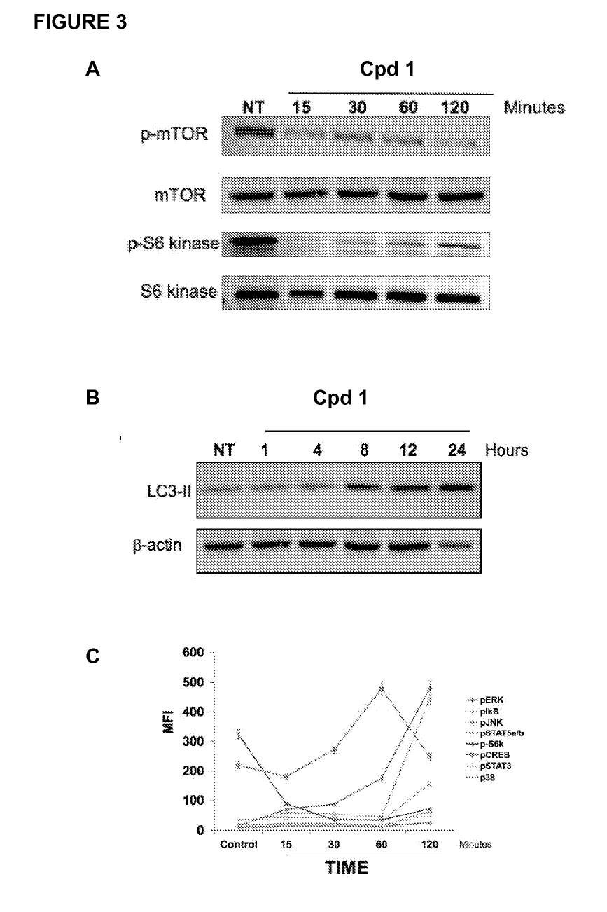

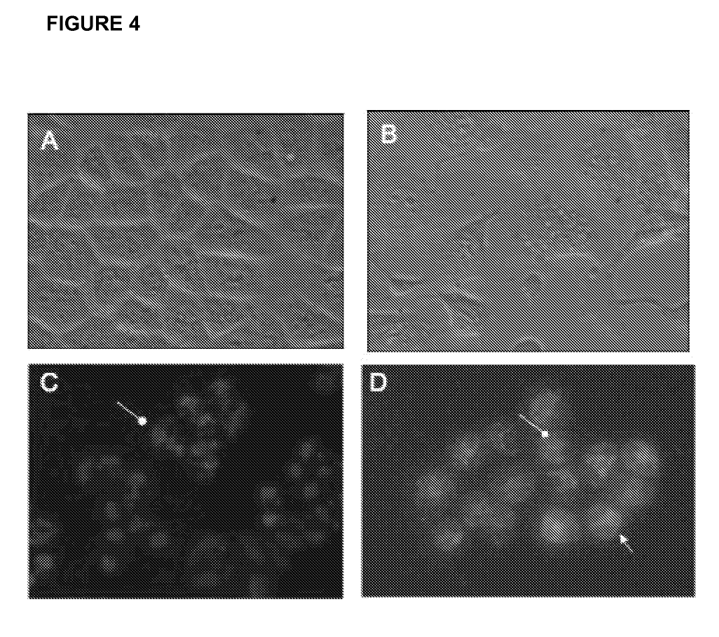

Morphologically, Compound 1 treated EOC cells contained large intracellular vacuoles (FIG. 4B) that stained positively with acridine orange (data not shown) thereby suggesting that Compound 1 induces autophagic cell death. To determine whether the process of autophagy is involved, the levels of phosphorylated mTOR (p-mTOR), a major regulator of the autophagic pathway, and one of its targets, ribosomal p70 S6 kinase (p-S6k) were determined. In addition, the levels of the autophagic marker LC3-II were determined. Western blot analyses showed that p-mTOR and p-S6k were down-regulated 15 mins post-Compound 1 treatment (FIG. 3A) followed by a significant increase in the levels of LC3-II (FIG. 3B) confirming the activation of the autophagic pathway.

To determine if the process of autophagy is the primary mechanism of Compound 1-induced cell death, cells were treated with Compound 1 (10 μg/ml) in the presence or absence of the well-characterized autophagy inhibitor, 3-methyladenine (3-MA), which inhibits the earliest step in the autophagosome formation (Seglen and Bohley, 1992). Cell viability studies employing 3 MA (10 μM) showed that the compound is not able to inhibit Compound 1-induced cell death (FIG. 2C). These data suggest that while some characteristics of autophagy were observed, it is not the primary mechanism of cell death induced by Compound 1.

To determine if Compound 1 affects specific survival pathways, a panel of phospho-proteins in cells incubated in the presence or absence of 10 μg/ml of Compound 1 was evaluated. As shown in FIG. 3C, of the 8 phospho-proteins tested, Compound 1 down-regulates only p-S6k, confirming the western blot results, and had minimal or no effect on pIκB, pSTAT3, and p38. Up-regulation of ERK, pJNK, pSTAT5a/b, and pCREB was also observed, which probably represents a compensatory response. These results suggest a specific effect of Compound 1 on the mTOR pathway.

### Example 3

### Mitochondrial Depolarization, Mitochondrial Translocation and Nuclear Translocation

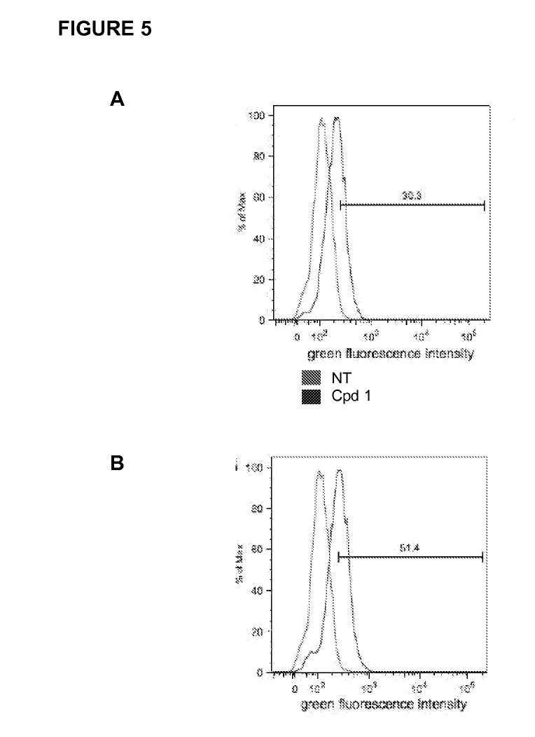

Control and treated cells were stained with JC-1 dye, a cationic fluorescent dye that stains intact mitochondria red and shifts to green during mitochondrial depolarization. When compared to vehicle control, immunofluourescent images of Compound 1 treated cells were mostly green in appearance (FIG. 4). This observed shift in JC-1 spectrum from red to green in Compound 1 treated cells confirms that Compound 1 is able to induce mitochondrial depolarization. This shift was confirmed by flow cytometry, which showed 30% (1 h) and 50% (4 h) of cells having depolarized mitochondria post-Compound 1 treatment (FIG. 5).

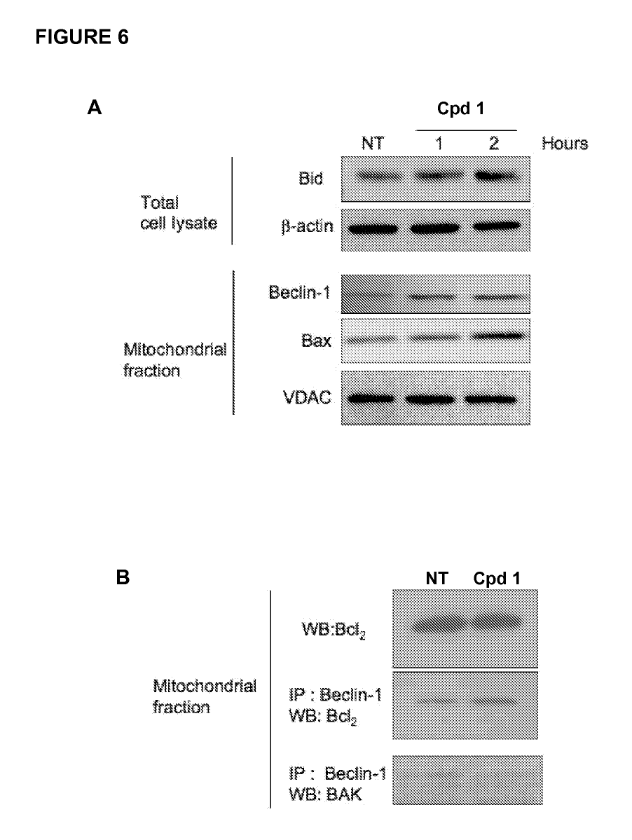

The stability of the mitochondrial membrane is partly controlled by the Bcl₂ family of proteins characterized by their conserved BH domains. Bid and Bax are cytoplasmic proteins that translocate to the mitochondria to initiate depolarization. In contrast, Bcl₂ is a resident mitochondrial protein that stabilizes the membrane. Treatment with Compound 1 induces mitochondrial translocation of Bax 2 h post-treatment but does not induce the activation of Bid (FIG. 6A). Given that Bax translocation, normally one of the first mediators of mitochondrial membrane destabilization, occurs after the onset of Compound 1-induced mitochondrial depolarization, an alternative mechanism must be responsible for the initiation of mitochondrial instability. Beclin-1 is a molecule best described in relation to its role in autophagy. In addition, Beclin-1 was recently shown to have a BH domain, therefore making it a justifiable member of the Bcl₂ family of proteins. Analysis of Beclin-1 mRNA by RT-PCR and Beclin-1 protein levels from whole cell lysates showed no changes in Beclin-1 message or protein expression after Compound 1 treatment (data not shown). However, analysis of mitochondrial fractions showed that Beclin-1 translocates to the mitochondria as early as 1 h post-Compound 1 treatment (FIG. 6A), which correlates with the onset of mitochondrial depolarization.

Apart from its role in the initiation of autophagy where it associates with Class III PI-3 kinase, it has been demonstrated that the BH3 domain of Beclin-1 is able to interact with both Bcl₂ and Bclₓₗ. In lieu of this, the inventors hypothesized that Beclin-1 mitochondrial translocation can lead to its interaction with Bcl₂ and therefore interfere with the ability of Bcl₂ to stabilize mitochondria. To show this association, Beclin-1 was immunoprecipitated from the mitochondrial fraction of untreated cells or cells treated with Compound 1 for 1 hr. The presence of Bcl₂ and Bak in the immunecomplex was then determined by western blotting. Higher levels of Bcl2 in complex with Beclin-1 were observed after Compound 1 treatment compared to the no treatment control (FIG. 6B) demonstrating that Compound 1-induced Beclin-1 mitochondrial translocation leads to Beclin1-Bcl₂ interaction. Bak was not observed in the complex suggesting that this is a specific interaction between Beclin-1 and Bcl2. These findings imply that Beclin-1 may have the ability to initiate mitochondrial depolarization by translocating to the mitochondria where it binds to and inactivates Bcl₂, potentially in a similar manner as those described for the Bid-Bak or Bid-Bax interaction.

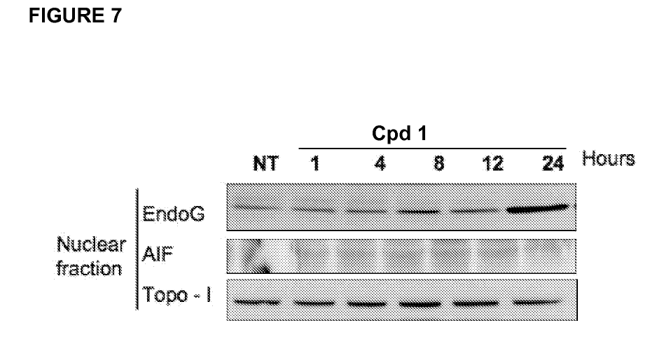

Mitochondrial nucleases represent a family of proteins that, once released from the mitochondria, can translocate to the nucleus and induce DNA fragmentation. The observation that Compound 1 is able to induce mitochondrial depolarization suggests that a mitochondrial nuclease may be responsible for the observed DNA fragmentation. Thus, western blot analysis was used to quantify the level of the mitochondrial nucleases, apoptosis-inducing factor (AIF) and endonuclease G (EndoG), in nuclear fractions of Compound 1-treated cells. An increase in the level of nuclear EndoG, but not AIF, was observed after Compound 1 treatment (FIG. 7) suggesting that EndoG is released from the mitochondria and translocates to the nucleus where it catalyses DNA fragmentation.

### Example 4

### Anti-Tumor Activity of Compound 1 on a Chemo-Resistant EOC Xenograft Model

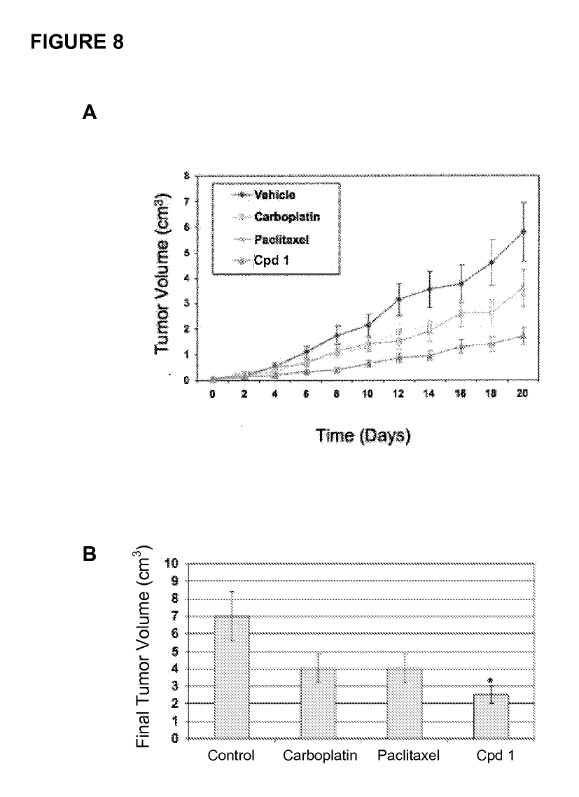

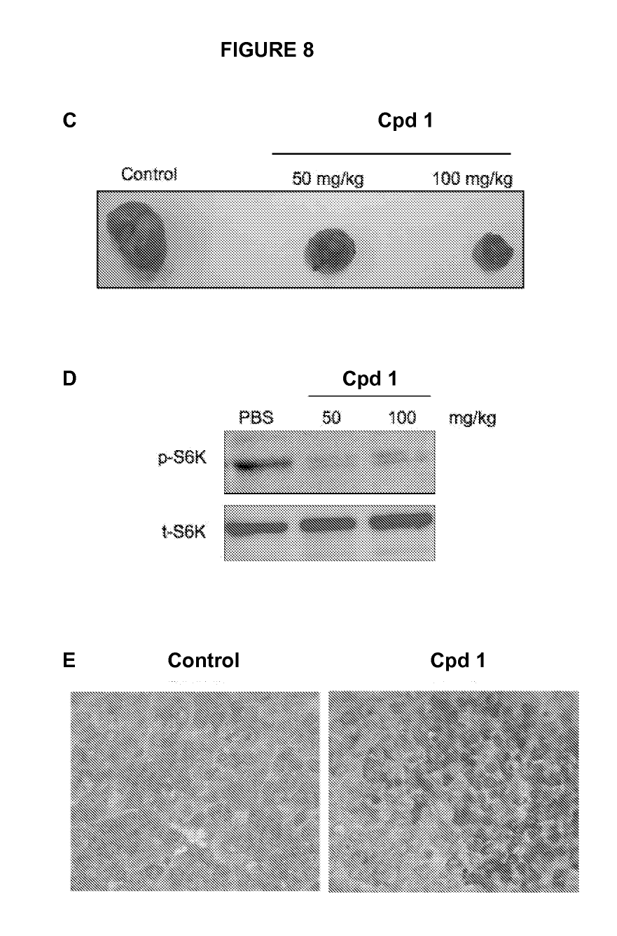

The inventors then assessed the in vivo anti-tumor activity of Compound 1. A nude mouse EOC xenograft model was established using EOC cells isolated from malignant ovarian cancer ascites. The anti-tumor activity of Compound 1 was compared with that of carboplatin and paclitaxel as described above. Compound 1 induced a significant decrease in tumor proliferation kinetics (FIG. 8A), and final tumor size and volume in a dose-dependent manner (FIGS. 8B and 8C), compared to carboplatin and paclitaxel (FIGS. 8A and 8B). T/C values were 30%, 58%, and 58% for Compound 1, carboplatin, and paclitaxel, respectively. No toxic side effects were noted in the Compound 1-treated groups, whereas mice in the carboplatin group exhibited severe weight loss and cachexia.

Finally, to demonstrate that the in vivo anti-tumor activity of Compound 1 parallels the observed mechanisms in vitro, tumors were lysed and the level of p-S6k was determined by western blot analysis and paraffin-embedded sections of mouse tumors were immunostained for EndoG. As shown in FIG. 8D, tumors taken from animals treated with Compound 1 had a significant decrease on p-S6k, compared to the vehicle control (FIG. 8D). In addition, the nuclear localization of EndoG in tumors taken from animals treated with Compound 1 was observed, while tumors from control animals had only cytoplasmic staining (FIG. 8E).

### Example 5

### Caspase-Independent Apoptosis Induced by Compound 10

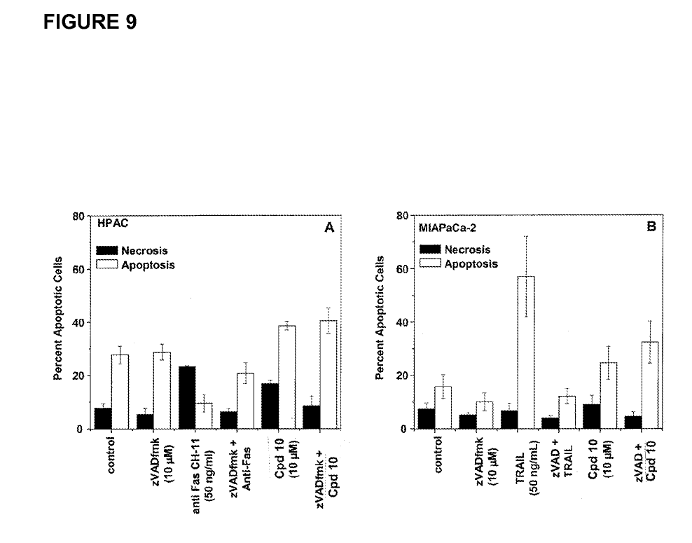

To determine whether Compound 10 was able to induce apoptosis in pancreatic cancer cells, the inventors conducted dose-escalating studies over 48 hr exposure and employed FACS analysis on Annexin-V-FITC/PI stained cells. In all cell types analysed (HPAC, MIAPaCa-2 and PANC-1), Compound 10 induced apoptosis in a dose-dependent fashion (FIG. 9). Very little necrosis was observed.

HPAC cells are resistant to TRAIL and sensitive to FasL-dependent apoptosis while MIAPaCa-2 cells are sensitive to TRAIL-dependent apoptosis and resistant to FasL-dependent apoptosis, both of which are caspase-dependent apoptosis pathways. As a result, Fas-activating antibody CH11 (Upstate) and TRAIL were used as positive controls respectively in these cell types. In HPAC cells, pre-treatment of cells with 10 μM caspase inhibitor I (zVAD-fmk) for 1 hr resulted in an inhibition of Fas-dependent necrosis while the same pre-treatment inhibited Compound 10-dependent necrosis but did not inhibit Compound 10-induced apoptosis (FIG. 9A). In MIAPaCa-2 cells, pre-treatment of cells with zVAD-fmk resulted in an inhibition of TRAIL-dependent apoptosis while the same pre-treatment inhibited Compound 10-dependent necrosis and apparently enhanced Compound 10-induced apoptosis, perhaps as a result of the cells being blocked from advancing to necrosis (FIG. 9B). These results show that a large component of the apoptotic effect induced by Compound 10 uses caspase-independent pathways in pancreatic cancer cells while the Compound 10-induced necrotic pathways are caspase-dependent.

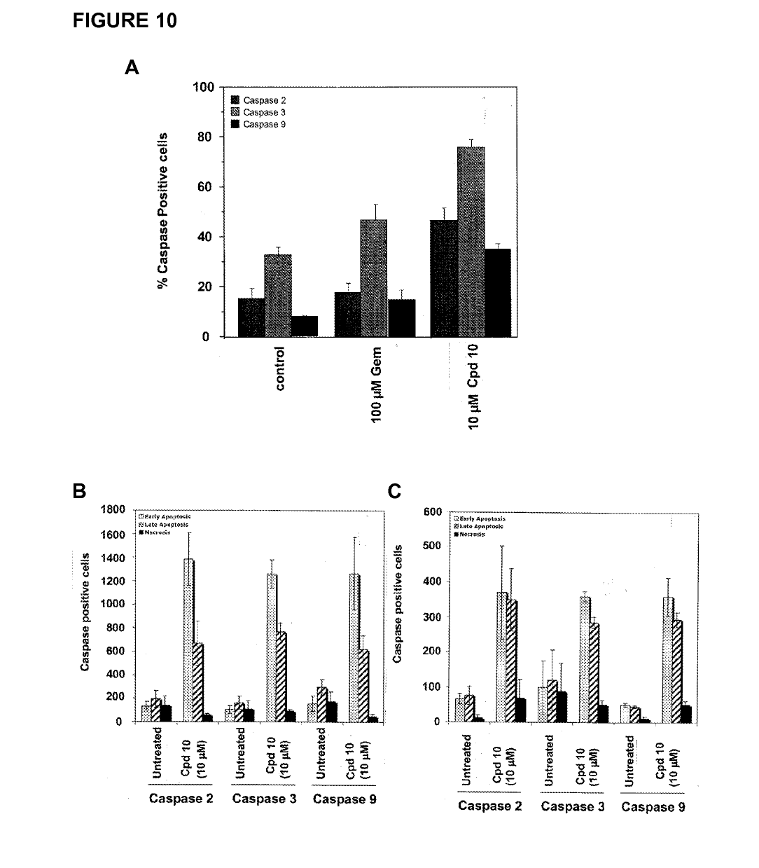

Caspase activation, apoptosis and necrosis were measured simultaneously in untreated and Compound 10-treated pancreatic cancer cells by flow cytometry. After 48 hrs, Compound 10 activated caspase 2, 3 and 9 in MIAPaCa-2 and PANC-1 cells (FIG. 10). Caspase positive cells were also Annexin-V-Cy5 positive showing that the Compound 10-induced caspase activation was in cells undergoing apoptosis. Approximately 60% of Compound 10-treated, caspase positive cells were Annexin-V positive and sytox blue negative indicating that they were in the early stages of apoptosis. Approximately 30% of the caspase positive cells bound both Annexin-V-Cy5 and sytox blue indicating that these cells were entering the later stages of aspoptosis. Less than 5% of the Compound 10-induced, caspase positive cells were sytox blue positive and Annexin-V negative indicating that caspases contibute little to Compound 10-dependent necrosis. With the exception of caspase-3 which was modestly induced in treated HPAC cells, neither caspase 2 or caspase 9 were induced in this cell line using the conditions employed (FIG. 10). At a higher concentration, (30 μM) Compound 10 activated caspase 9 but not caspase 2 in HPAC cells (data not shown). These data suggest that caspase-independent mechanisms of apoptosis are activated in Compound 10 treated HPAC cells.

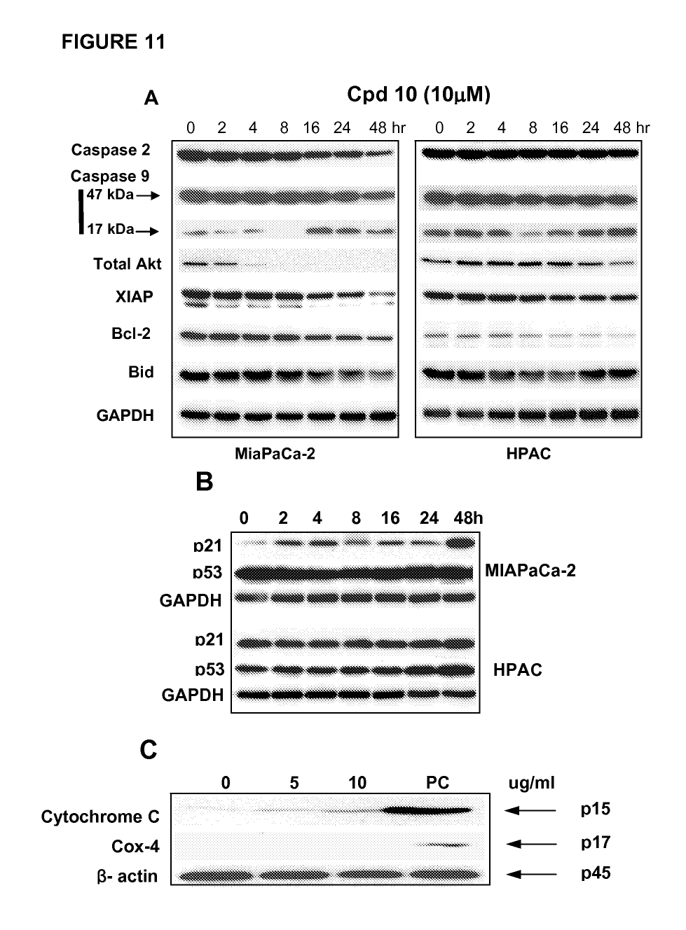

Western blot analysis showed that full-length Caspase 2 (48Kd) was cleaved in MIAPaCa-2 cells by exposure to 10 μM Compound 10 (FIG. 11A). In contrast, Caspase 2 was cleaved to a lesser extent by Compound 10 in HPAC cells (FIG. 11A). Over the course of the time-course experiment, Compound 10 induced an initial drop in cleaved caspase 9 (17kd) followed by an increase after 16 hr in both cell types (FIG. 11B). Treatment with Compound 10 did not cause significant cleavage of Caspase 3 in MIAPaCa-2 and HPAC cells. Longer exposure of the same blots showed minor cleavage of Caspase 3 in HPAC but not in MIAPaCa-2 cells (data not shown). Taken together, these experiments suggest that Compound 10 is affecting both caspase activation and synthesis.

### Example 6

### Effect of Compound 10 and on In Vitro Mitochondrial Potential and Cell Cycle Progression

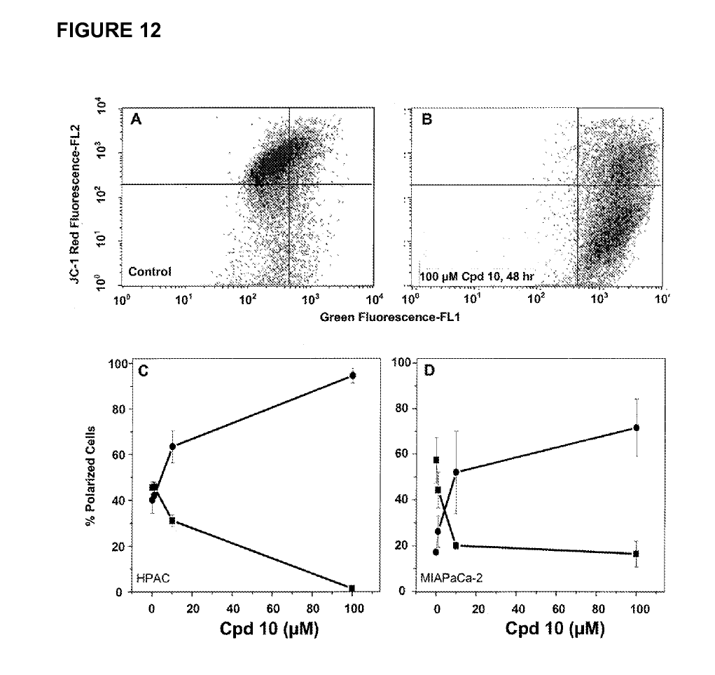

In all cell lines tested (see Example 5), Compound 10 depolarized the mitochondria of these cells in a dose-dependent manner following 48 hrs of treatment showing that the intrinsic pathway of apoptosis has been activated (FIG. 12).

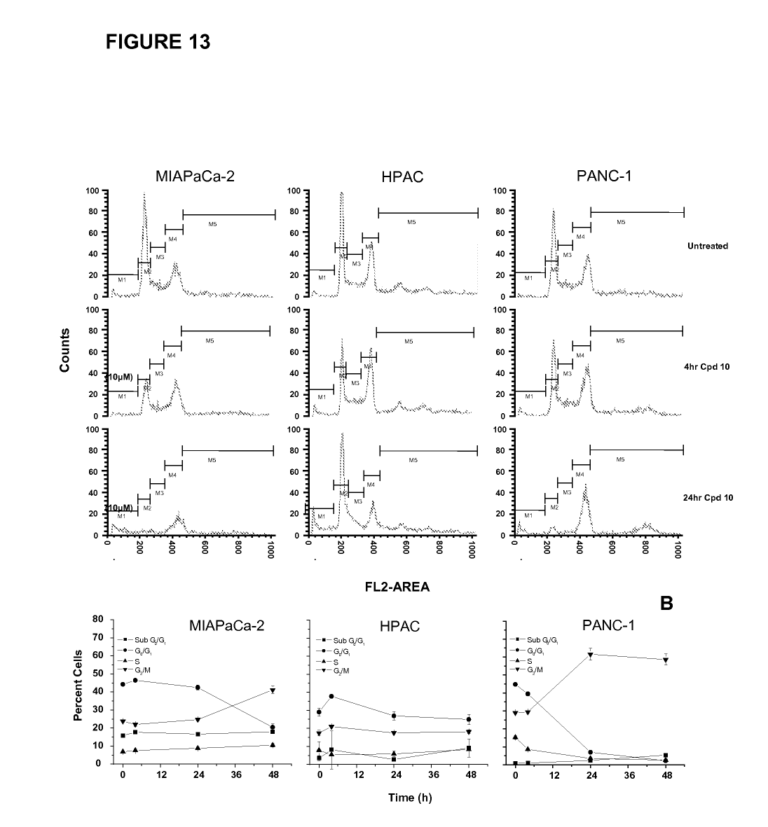

In all three cell lines Compound 10 arrested the cell cycle in G₂/M phase to varying degrees, with a loss of G₀/G₁ and an increase in sub-G₀/G₁ phase (FIG. 13). The timing of Compound 10 induced G₂/M arrest was different for each cell line. PANC-1 was arrested in G₂/M some 4-24 hr post Compound 10 treatment and remained in arrest for at least 48 hrs. MIAPaCa-2 cells entered G₂/M arrest some 24-48 hr post Compound 10 treatment. Accompanying the arrest was a corresponding decrease in the G₀/G₁ cell population. The G₂/M arrest observed in HPAC cells in response to Compound 10 was less pronounced and was accompanied with an arrest in G₀/G₁. However, the arrest induced would appear to be transient (FIG. 13).

Previous studies on OEC cells had shown that the caspase 3 inhibitor XIAP is significantly reduced by phenoxodiol and that this isoflavanoid stimulates auto-degradation of this BIRC family member (Alvero et al., 2006). In both HPAC and MIAPaCa-2 cells, Compound 10 reduced the level of XIAP (FIG. 11A), however XIAP degradation was more pronounced in MIAPaCa-2 cells. Reduction of XIAP is likely to remove a block on caspase 3 activation (Deveraux et al. 1997). Importantly, total Akt is also reduced at an early timepoint (4 hr) suggesting that total Akt removal accompanied by XIAP degradation are two of the earliest events in Compound 10 mechanism of action. Reduction in Akt expression would also impact on the expression of XIAP (Alvero et al., 2006 and Alvero et al., 2008).

### Example 7

### Effect of Compound 10 on Bcl₂ Family Member Stoichiometry

As noted in Example 6, Compound 10 induced mitochondrial depolarization. The inventors therefore investigated whether its action may be involved in altering the stoichiometry of pro-apoptotic (Bid, Bak, Bax) and anti-apoptotic (Bcl₂, Bcl_{XL}) Bcl₂ family members. Levels of Bcl₂ were lowered by Compound 10 treatment in MIAPaCa-2 and HPAC cells (FIG. 11A). Treatment with either Compound 10 induced Bid cleavage over time in MIAPaCa-2 cells (FIG. 11A) while there appeared to be a transitory increase in Bid cleavage by Compound 10 in HPAC cells (FIG. 11A).

The cell cycle is controlled by cyclin-dependent kinases, which in turn are regulated by p21, usually in a p53-dependent pathway. Most pancreatic cancers have mutant p53, which arrests p53-dependent transcription. HPAC cells are unusual in having a wild type p53, while MIAPaCa-2 and PANC-1 have R₂₄₈W and R₂₇₃H mutations in p53. These mutations are in loop 3 of p53 and affect the ability of p53 to bind DNA. The basal level of p53 protein is 94× higher in MIAPaCa-2 cells than HPAC cells (data not shown). Expression of p53 in MIAPaCa-2 cells was unaffected by Compound 10 (FIG. 11B) but was elevated by 48 hr in HPAC cells (FIG. 11B). p21 is vanishingly low in MIAPaCa-2 cells and is elevated by Compound 10 (FIG. 11B), while p21 is abundant in HPAC cells and is essentially unchanged by Compound 10 (FIG. 11B).

### Example 8

### Anti-Tumor Activity of Compound 10 Alone and in Combination with Gemcitabine

For xenograft studies, five week old male Balb/c nude mice were maintained on an isoflavone-free diet to remove background isoflavone levels contributed by standard feed. On the day of inoculation a suspension of MIAPaCa2 or HPAC cells was prepared in DMEM (-FBS), chilled on ice and then an equal volume of matrigel (BD) was added. Mice were inoculated subcutaneously with 3×10⁶ MIAPaCa2 or HPAC cells bilaterally (midway between the axillary and inguinal region) along the dorsal surface, ensuring that cells to be injected were growing at mid-end log phase. Tumor growth was monitored over 10-15 days. Compound 10 was given orally by gavage in 1% carboxy methyl cellulose (CMC) as a vehicle. Gemcitabine was injected intraperiponeally in PBS. In combination groups animals were first dosed with Compound 10 and then dosed with gemcitabine using the respective administration schedule, dosage and route of delivery stated.

In the HPAC model of pancreatic cancer, animals dosed with the Compound 10 and gemcitabine combination at 50 and 4 mg/kg respectively displayed significantly reduced rates of tumor proliferation and terminal tumor burden (% T/C=38) in comparison to the respective single agent controls (data not shown). Further, those animals receiving the combination Compound 10 and gemcitabine at dosages half that of the single agent controls (25 mg/kg Compound 10 and 2 mg/kg gemcitabine) also had significantly reduced tumor proliferation kinetics and reduced terminal tumor burden (% T/C=47.8) when compared to monotherapy controls. The effectiveness of both Compound 10:gemcitabine dosage combinations was further demonstrated by the observation that tumors on animals dosed with either combination proliferated at significantly reduced rates in a dose responsive manner when compared to the respective monotherapy controls (data not shown). Together these data indicate that Compound 10 and gemcitabine when dosed in combination act synergistically in reducing overall tumor burden using the HPAC pancreatic cancer tumor model.

In the MIAPaCa-2 model of pancreatic cancer, when compared to vehicle control, orally administered Compound 10 (100 mg/kg, qd×12) retarded the rate of tumor proliferation, at a level similar to that of gemcitabine (20 mg/kg, q3d×4). Animals dose dosed with both Compound 10 and gemcitabine at 100 and 20 mg/kg respectively had significantly reduced tumor growth and terminal tumor burden (% T/C=51) when compared to vehicle and single agent controls. The observed mean terminal tumor burden for the combination was markedly below that of the respective single agent controls (100 mg/kg Compound 10, T/C=94%; for 20 mg/kg gemcitabine, T/C=79%. These data suggest that when dosed in combination, Compound 10 and gemcitabine may act synergistically to reduce overall tumor burden using the MIAPaCa-2 pancreatic cancer xenografts.

In those animals receiving high and low dose combinations of gemcitabine and Compound 10, all markers of renal and hepatic function were comparable to that of control animals as were white blood cell and red blood cell counts (data not shown). Histopathological analysis of vital organs revealed no abnormalities that could be attributed to drug toxicity (not shown). These data demonstrate that Compound 10 does not exacerbate gemcitabine toxicity with respect to the parameters measured and can be used safely in combination with gemcitabine at the dosages employed.

### REFERENCES

Ahn E-Y, et al., Am J. Pathol. 2003 163: 2053-2063.
Alvero A B, et al. Molecular mechanism of phenoxodiol-induced apoptosis in ovarian carcinoma cells. Cancer 2006 Feb. 1; 106 (3): 599-608.
Alvero A B, et al. Anti-tumour activity of phenoxodiol: From bench to Clinic. Future Oncology 2008 (accepted).
Behrens B C, et al. Characterization of a cis-diamminedichloroplatinum(II)-resistant human ovarian cancer cell line and its use in evaluation of platinum analogues. Cancer Res 1987 Jan. 15; 47 (2): 414-418.
Deveraux Q L, et al. X-linked IAP is a direct inhibitor of cell-death proteases. Nature 1997 388: 300-304.
Hail Jr N, et al. Apoptosis effector mechanisms: A requiem performed in different keys. Apoptosis 2006; 11: 889-904.
Hay N and Sonenberg N. Upstream and downstream of mTOR. Journal Genes & Dev 2004; 18: 1926-1945.
Kamsteeg M, et al. Phenoxodiol—an isoflavone analog—induces apoptosis in chemoresistant ovarian cancer cells. Oncogene 2003 May 1; 22 (17): 2611-2620.
Kelly M G, et al. TLR-4 signaling promotes tumor growth and paclitaxel chemoresistance in ovarian cancer. Cancer Res 2006 Apr. 1; 66 (7): 3859-3868.
Kluger H M, et al. The X-linked inhibitor of apoptosis protein (XIAP) is up-regulated in metastatic melanoma, and XIAP cleavage by Phenoxodiol is associated with Carboplatin sensitization. J Transl Med 2007; 5: 6.
Seglen P O and Bohley P. Autophagy and other vacuolar protein degradation mechanisms. Experientia 1992 Feb. 15; 48 (2): 158-172.

1. A method for inducing or promoting caspase-independent apoptosis in a cell, the method comprising exposing to the cell an effective amount of a compound of formula (I) 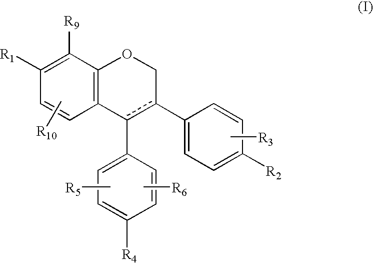
wherein
R₁ is hydrogen, hydroxy, alkyl, alkoxy, halo or OC(O)R₇,
R₂ and R₃ are independently hydrogen, hydroxy, alkoxy, alkyl, cycloalkyl, halo or OC(O)R₇,
R_{4,} R₅ and R₆ are independently hydrogen, hydroxy, alkoxy, alkyl, cycloalkyl, acyl, amino, C₁₋₄-alkylamino or di(C₁₋₄-alkyl)amino, OC(O)R₇ or OR₈,
R₇ is hydrogen, alkyl, cycloalkyl, aryl, arylalkyl or amino, and
R₈ is aryl or arylalkyl,
R₉ and R₁₀ are independently hydrogen, hydroxy, alkyl, alkoxy or halo, and the drawing [symbol] represents a single bond or a double bond,
or a pharmaceutically acceptable salt or derivative thereof.

2. The method of claim 1 wherein the cell is not a cancer cell.

3. The method of claim 1 wherein the cell is selected from a myocardial cell or an immune cell.

4. The method of claim 1 wherein the compound is 3-(4-hydroxyphenyl)-4-(4-methoxyphenyl)-8-methylchroman-7-ol, with the structure:

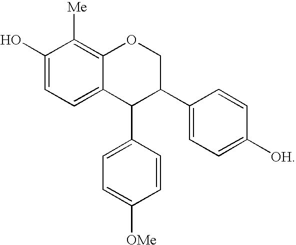

5. The method of claim 1 wherein the compound is 3-(4-hydroxyphenyl)-4-(4-methoxyphenyl)chroman-7-ol, with the structure:

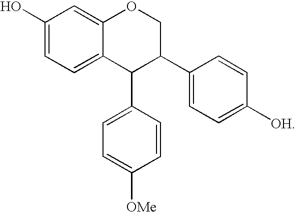

6. A method for inhibiting mTOR activity in a cell, the method comprising exposing to the cell an effective amount of a compound of formula (I) as described herein.

7. The method of claim 6 wherein the inhibition of mTOR activity comprises dephosphorylation of mTOR.

8. A method for the treatment or prevention of a disease or condition, the method comprising administering to a subject in need thereof an effective amount of a compound of formula (I) as described herein, or a pharmaceutically acceptable salt or derivative thereof, optionally in association with one or more pharmaceutically acceptable diluents, adjuvants and/or excipients, wherein the compound induces or promotes caspase-independent apoptosis and/or inhibits mTOR activity in at least one cell of the subject.

9. The method of claim 8 wherein the cell is not a cancer cell.

10. The method of claim 8 wherein the cell is a proliferating T cell.

11. The method of claim 8 wherein the disease or condition is associated with aberrant or otherwise unwanted cell growth or proliferation.

12. The method of claim 8 wherein the disease or condition is selected from stenosis, restenosis, transplant rejection, rheumatoid arthritis, a T cell leukemia, an autoimmune disease, and a transplant or graft rejection.

13. The method of claim 12 wherein for the treatment of stenosis or restenosis the compound or a composition comprising the compound is coated onto or otherwise incorporated into a stent for introduction into a coronary artery.

14. The method of claim 13 wherein the stent is such that the compound or composition is eluted from the stent over a period of time so as to achieve the desired outcome.

15. The method of claim 12 wherein the autoimmune disease is selected from cirrhosis, psoriasis, lupus, rheumatoid arthritis, Addison's disease, infectious mononucleosis, Sézary's syndrome and Epstein-Barr virus infection.

16. The method of claim 8 wherein the compound is seletcted from 3-(4-hydroxyphenyl)-4-(4-methoxyphenyl)-8-methylchroman-7-ol, with the structure: 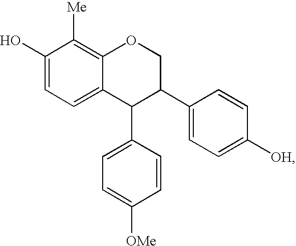
and 3-(4-hydroxyphenyl)-4-(4-methoxyphenyl)chroman-7-ol, with the structure:

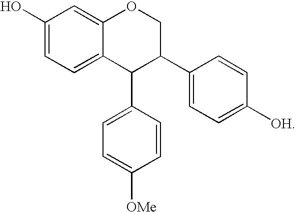

17. An agent for the treatment or prevention of a disease or condition associated with aberrant or otherwise unwanted cell growth and/or proliferation, the agent comprising a compound of formula (I) as described herein, or a pharmaceutically acceptable salt or derivative thereof.

18. An implantable medical device for delivering at least one active agent to a cell or tissue in a subject, wherein the at least one active agent comprises a compound of formula (I) as described herein.

19. The medical device of claim 18 wherein the compound is coated onto or otherwise incorporated into the device for administration of the compound to the cell or tissue.

20. The medical device of claim 18 wherein the device is a drug-eluting stent.
